(19) United States
(12) Patent Application Publication
(10) Pub. No.: US 2022/0401394 A1
(43) Pub. Date: Dec. 22, 2022

(54) ORAL TABLETS COMPRISING ROLLER-COMPACTED GRANULES OF NAPROXEN SODIUM, METHODS OF PREPARING THEREOF, AND METHODS OF USING THEREOF

(71) Applicant: Bayer HealthCare LLC, Whippany, NJ (US)

(72) Inventors: Satish KAMATH, Budd Lake, NJ (US); Rachan SIRIHORACHAI, Cranbury, NJ (US); Karthik Yadav JANGA, Parsippany, NJ (US)

(73) Assignee: Bayer HealthCare LLC, Whippany, NJ (US)

(21) Appl. No.: 17/786,728

(22) PCT Filed: Dec. 18, 2020
(86) PCT No.: PCT/US2020/066187

§ 371 Date: Jun. 17, 2022

Related U.S. Application Data
(60) Provisional application No. 62/950,196, filed on Dec. 19, 2019.

(51) Int. Cl. A61K 31/192; A61K 9/20; A61K 9/24; A61K 31/167

(52) U.S. Cl. CPC A61K 31/192; A61K 9/2018; A61K 9/2009; A61K 9/2059; A61K 9/209; A61K 31/167; A61K 9/2054

(57) ABSTRACT

The present disclosure relates to oral naproxen sodium tablets comprising roller-compacted granules, methods of preparing thereof, and methods of using thereof. The naproxen sodium tablets are formulated for and prepared by dry granulation methods, specifically roller compaction. The combination of dry granulation compatible excipients with roller compaction methods results m naproxen sodium tablets that exhibit an enhanced dissolution profile and shorter disintegration time as compared to commercially available oral naproxen sodium tablets prepared by standard wet granulation methods.

### CROSS-REFERENCE TO RELATED APPLICATION

This application claims the benefit of U.S. Provisional Application No. 62/950,196, filed on Dec. 19, 2019, which is hereby incorporated by reference in its entirety.

### FIELD

The present disclosure relates generally to oral tablets prepared by dry granulation methods and, more specifically, to naproxen sodium tablets with enhanced dissolution profiles and disintegration times, dry granulation methods for preparing the naproxen sodium tablets, and methods of using the naproxen sodium tablets.

### BACKGROUND

Naproxen sodium is a non-steroidal anti-inflammatory drug (NSAID) used to treat inflammation associated with a variety of conditions as well as to provide long-lasting relief from mild to moderate pain. Although naproxen sodium is most commonly sold in oral tablets for immediate release, immediate release formulations of naproxen sodium may exhibit a delayed onset of therapeutic action (e.g., up to an hour) after administration. Naproxen sodium tablets having improved properties, such as greater initial dissolution rates and/or shorter disintegration times, could potentially provide more rapid onset of action. Naproxen sodium tablets having an earlier onset of therapeutic action could subsequently lead to faster alleviation of inflammation and pain, which would be desirable for consumers.

Compositions of commercially available naproxen sodium tablets have remained largely unchanged over several decades due to their compatibility with wet granulation methods, which produce tablets having consistent, well-established physicochemical properties. However, despite their reproducibility, wet granulation methods include numerous process steps that can result in significant inefficiencies and substantial loss of material throughout production unless optimized for operating equipment and conditions specific to individual manufacturing plants. Moreover, the various stages and procedures associated with wet granulation methods often involve immense costs for installation of new or updated equipment to increase efficiency or output capacity.

Growing global demand for naproxen sodium has renewed interest in the search for improved formulations as well as alternative methods to increase manufacturing capacity and efficiency over existing methods. The difficulty of developing new methods and/or formulations lays in the fact that new process methods for manufacturing the formulations must be equally as cost effective and reliable as existing methods, while the new formulations must have equivalent or superior drug release and bioavailability profiles as compared to the original formulations. Although alternative formulations of naproxen sodium tablets and processes for manufacturing them have been investigated in the past, none has been successful in supplanting existing formulations and the associated wet granulation methods at commercial scale.

The difficulty in devising cost-effective alternative methods for manufacturing naproxen sodium tablets also presents a barrier to the development of new fixed-dose combinations that might include naproxen as one of the key active pharmaceutical ingredients. The preparation of oral tablets containing naproxen sodium along with other active ingredients further complicates cost and manufacturing considerations by adding constraints on excipients based on the physical and chemical compatibility requirements of the both the naproxen and other actives. Consumers also expect fixed-dose combinations to deliver the same or superior drug release and bioavailability profile for the additional active ingredients as compared to their standalone formulations. Notwithstanding any manufacturing constraints, identifying a viable formulation that will satisfy these drug release and bioavailability criteria is a non-trivial task for active ingredients possessing different dissolution and disintegration properties and/or storage stability requirements. For tablets containing naproxen sodium, where manufacturing considerations are significant, there is even less flexibility in modulating excipients to arrive at a satisfactory fixed-dose combination formulation.

Thus, there remains a need for both improved formulations for oral tablets of naproxen sodium either as the sole active pharmaceutical ingredient or in combination with additional active pharmaceutical ingredients, and improved methods for their production.

### BRIEF SUMMARY

The present disclosure provides naproxen sodium tablets having an enhanced dissolution profile (e.g., greater initial dissolution rates) and shorter disintegration times than existing formulations. The improved properties of the naproxen sodium tablets can be achieved by a unique composition of excipients in combination with dry roller compaction methods to provide granules within the tablets that are highly penetrable to dissolution media (e.g., water) than existing formulations. The faster dissolution and shorter disintegration times of the naproxen sodium tablets may result in faster onset of therapeutic action for reducing inflammation and pain.

In addition to conferring improved properties to the naproxen sodium tablets, the methods of preparing naproxen sodium tablets as provided herein may also allow for simpler and more efficient manufacture than existing wet granulation methods, by using less excipient material overall, reducing the total number of process steps required, and producing tablets having consistent dissolution and disintegration profiles for a range of various process parameters. These processes may be employed for the preparation of oral tablets containing naproxen sodium as the lone active ingredient or in combination with other active pharmaceutical ingredients.

In one aspect, provided herein is a naproxen sodium tablet, comprising:
granules comprising naproxen sodium;
mannitol;
colloidal silicon dioxide;
one or more lubricants; and
one or more superdisintegrants,
   wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.

In another aspect, provided herein is a naproxen sodium tablet, comprising:
granules comprising naproxen sodium;
mannitol;
colloidal silicon dioxide;
stearic acid;
sodium starch glycolate; and
magnesium stearate,
   wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.

In yet another aspect, provided herein are methods of preparing a naproxen sodium tablet as described herein, comprising:

combining naproxen sodium, mannitol, colloidal silicon dioxide, one or more lubricants and one or more superdisintegrants to provide a blend mixture;

compacting the blend mixture by roller compaction to provide ribbons;

milling the ribbons to provide granules;

combining the granules with mannitol, one or more lubricants, one or more superdisintegrants, and optionally colloidal silicon dioxide, to provide a tableting mixture; and

compressing the tableting mixture to provide the naproxen sodium tablet.

In still yet another aspect, provided herein are methods of preparing a naproxen sodium tablet as described herein, comprising:

combining naproxen sodium, mannitol, colloidal silicon dioxide, stearic acid and sodium starch glycolate to provide a blend mixture;

compacting the blend mixture by roller compaction to provide ribbons;

milling the ribbons to provide granules;

combining the granules with mannitol, sodium starch glycolate, magnesium stearate, and optionally colloidal silicon dioxide, to provide a tableting mixture; and

compressing the tableting mixture to provide the naproxen sodium tablet.

In yet a further aspect, provided herein is a method of treating pain or ache in a subject in need thereof, comprising administering a naproxen sodium tablet as described herein to the subject.

In still another aspect, provided herein is a method of reducing fever in a subject in need thereof, comprising administering a naproxen sodium tablet as described herein to the subject.

The present disclosure also provides bilayer naproxen sodium tablets that combine naproxen sodium and one or more additional active pharmaceutical ingredients, such as acetaminophen. More specifically, the present disclosure provides fixed-dose combination bilayer tablets comprising roller-compacted granules of naproxen sodium in a primary layer and acetaminophen in a secondary layer. The bilayer tablets provided herein pair complementary disintegration mechanisms of naproxen sodium and acetaminophen with the unique composition of the roller-compacted granules comprising naproxen sodium described above to provide a formulation that exhibits surprisingly shorter disintegration times than existing tablet formulations containing naproxen sodium as the sole active pharmaceutical ingredient.

In another aspect, provided herein is a bilayer naproxen sodium tablet, comprising:

a primary layer, comprising:
granules, comprising naproxen sodium;
mannitol;
colloidal silicon dioxide;
one or more binders;
one or more lubricants; and
one or more superdisintegrants, and

a secondary layer, comprising:
one or more additional active pharmaceutical ingredients;
colloidal silicon dioxide;
one or more binders;
one or more lubricants; and
one or more superdisintegrants,
   wherein the tablet has a disintegration time of less than 5 minutes as determined by the USP Disintegration Test in water using a basket-rack assembly with disks at 37° C.±0.5° C.

In yet another aspect, provided herein is a bilayer naproxen sodium tablet, comprising:
a naproxen sodium layer, comprising:
   granules, comprising naproxen sodium;
   mannitol;
   colloidal silicon dioxide;
   sodium starch glycolate;
   starch and/or partially pregelatinized starch;
   stearic acid or magnesium stearate; and
   croscarmellose sodium, and
an acetaminophen layer, comprising:
   acetaminophen;
   colloidal silicon dioxide;
   starch and/or partially pregelatinized starch;
   stearic acid or magnesium stearate, and
   croscarmellose sodium,
      wherein the tablet has a disintegration time of less than 5 minutes as determined by the USP Disintegration Test in water using a basket-rack assembly with disks at 37° C.±0.5° C.

In a further aspect, provided herein is a method of treating pain or ache in a subject in need thereof, comprising administering a bilayer naproxen sodium tablet as described herein to the subject.

In still another aspect, provided herein is a method of reducing fever in a subject in need thereof, comprising administering a bilayer naproxen sodium tablet as described herein to the subject.

### DESCRIPTION OF THE FIGURES

The present application can be understood by reference to the following description taken in conjunction with the accompanying figures.

FIG. 1 depicts an exemplary process for the preparation of naproxen sodium tablets as described herein.

FIG. 2 depicts the dissolution profile of a naproxen sodium tablet prepared by dry roller compaction (with two different film coatings) as compared to the dissolution profile of a commercially available film-coated oral tablet of naproxen sodium prepared by wet granulation as determined by USP Dissolution Test Apparatus-2 in phosphate buffer of pH 7.4 at 37° C.±0.5° C.

FIG. 3 depicts the dissolution profile of a naproxen sodium tablet prepared by dry roller compaction (with two film coatings) as compared to the dissolution profile of a commercially available film-coated oral tablet of naproxen sodium prepared by wet granulation as determined by USP Dissolution Test Apparatus-2 in phosphate buffer of pH 5.8 at 37° C.±0.5° C.

FIG. 4 depicts an exemplary process for the preparation of bilayer naproxen sodium tablets as described herein.

FIGS. 5A-5E depict combination monolayer and bilayer tablets comprising naproxen sodium and acetaminophen at various time points during a comparative disintegration study. FIG. 5A shows a photograph of a monolayer oral tablet comprising naproxen sodium granules (prepared by dry roller compaction) and acetaminophen granules (commercially available) (at left) and an oral bilayer tablet comprising naproxen sodium granules (prepared by dry roller compaction) in the primary layer and acetaminophen granules in the secondary layer (at right). FIG. 5B-5E show photographs, illustrating the time elapsed disintegration of each formulation in a disintegration apparatus at zero (0) seconds (FIG. 5B), at 10 seconds (FIG. 5C), at 35 seconds (FIG. 5D), and at 3 minutes, 3 seconds (FIG. 5E).

FIGS. 6A-6B depict a comparison plot of disintegration times for various tablet formulations of naproxen sodium and acetaminophen.

### DETAILED DESCRIPTION

The present disclosure provides naproxen sodium tablets prepared by dry granulation, and, more specifically, naproxen sodium tablets comprising roller-compacted granules, wherein the naproxen sodium tablets may have an enhanced dissolution profile and shorter disintegration times than existing formulations. In one aspect of the present disclosure, the naproxen sodium tablets of the present disclosure comprise granules comprising naproxen sodium; mannitol; colloidal silicon dioxide; stearic acid; sodium starch glycolate; and magnesium stearate.

The naproxen sodium tablets comprising granules of naproxen sodium with the particular excipients described herein may display a superior dissolution profile as compared to that of commercially available naproxen sodium tablets. Specifically, the naproxen sodium tablets of the present disclosure can exhibit about 80% dissolution of naproxen sodium after ten minutes and nearly complete or complete dissolution (95% or greater) of naproxen sodium within twenty minutes, whereas naproxen sodium tablets prepared by existing wet granulation methods require at least twenty minutes to reach 80% dissolution and thirty minutes or longer to achieve complete dissolution.

The improved dissolution and disintegration properties of the tablets provided herein can be achieved by the use of select intragranular and extragranular excipients within the tablet. Naproxen sodium has been observed to disintegrate according to a surface erosion mechanism; its disintegration is not strongly influenced or accelerated by increasing the concentration of superdisintegrants present. However, for the tablets of the present disclosure, the particular choice and combination of extragranular excipients (mannitol, sodium starch glycolate, and optionally colloidal silicon dioxide) is believed to promote the uptake and transmission of dissolution media, such as water, into the core of the tablet. Similarly, the use of a select composition of intragranular excipients (mannitol, sodium starch glycolate, stearic acid, and colloidal silicon dioxide) may result in granules that are compactable into tablets but still reasonably porous to facilitate the passage of dissolution media, through the tablet and to penetrate larger granules. The combination of extragranular excipients with dry roller compaction methods provide naproxen sodium tablets that are more penetrable to dissolution media than existing formulations, thereby providing the enhanced dissolution and disintegration.

In addition to the particular naproxen sodium tablet formulation having enhanced properties, provided herein are also dry granulation methods for preparing the naproxen sodium tablets. The methods of the present disclosure involve dry granulation, specifically dry granulation by roller compaction, in contrast to commercially available oral tablets prepared by wet granulation. Both wet granulation and dry granulation involve the agglomeration or densification of powder materials to facilitate downstream processing. However, dry granulation methods achieve densification via direct physical compression whereas wet granulation methods employ granulation solvents or fluids to induce aggregation. The dry granulation-based methods of preparing the naproxen sodium tablets of the present disclosure involve first combining naproxen sodium with intragranular excipients as described above in a blend mixture. The blend mixture is passed through the roller compactors and the compacted material milled into porous granules. The granules are further combined with the extragranular excipients described above and compressed into a final tablet dosage form.

The use of the roller compaction methods provided herein ultimately can provide more efficient manufacture than existing wet granulation methods, by using less excipient material overall, reducing the number of production steps, improving manufacturing time, and producing tablets having consistent dissolution and disintegration profiles with minimal optimization of process parameters. For example, roller compaction methods may be readily adapted in either a continuous process or batch process unlike wet granulation methods, which are primarily carried out via batch processing. Batch processing is typically utilized for wet granulation methods to allow sufficient residence times to obtain desired granule size distributions and residual moisture contents. Additional benefits of roller compaction may include reduced upfront installation costs, flexibility to scale up and/or down production batches, and lower operational costs.

Moreover, it has been further unexpectedly observed that the enhanced dissolution profile of the naproxen sodium tablets achieved with the particular excipient combinations described herein could be obtained for a broad range of values for process parameters during dry granulation (roll speed, roll pressure, mill speed, etc.) and tableting (compression force). The dry granulation methods described herein have been observed not only to be compatible with the select combination of excipients described herein but also to allow for the formation of porous structure within the granules at surprisingly consistent levels of porosity and granule size distributions despite variation of roller compaction parameters. As such, the excipient composition of the naproxen sodium tablets may be described as highly compatible with roller compaction methods and results in tablets having enhanced dissolution with high reproducibility for a range of process parameters.

In another aspect, the present disclosure provides naproxen sodium tablets comprising granules comprising naproxen sodium, and one or more additional active pharmaceutical ingredients. Fixed-dose combinations comprising naproxen sodium and additional active pharmaceutical ingredients may provide additive or complementary therapeutic benefits due to different mechanisms of therapeutic action, different onsets of action, and different pharmacokinetic half-lives. For example, fixed-dose combinations comprising naproxen sodium and acetaminophen (or other analgesic) may provide complementary pain relieving and/or fever relieving effects. Naproxen sodium, a non-steroidal anti-inflammatory drug (NSAID), is widely used as a long-lasting analgesic (with a half-life between 8 and 12 hours) but suffers from a relatively slow onset of action. In contrast, acetaminophen is a non-NSAID analgesic, with a rapid onset of action (within 15 minutes) but a much shorter half-life (4 hours) and overall duration of pain relieving effect. The combination of naproxen sodium and acetaminophen in a single dosage form provides consumers with the dual advantages of rapid and long-lasting pain-relief.

In one aspect, the present disclosure provides a bilayer naproxen sodium tablet comprising a naproxen sodium layer, wherein the naproxen sodium layer comprises granules comprising naproxen sodium, as described herein, mannitol; colloidal silicon dioxide; sodium starch glycolate; starch and/or partially pregelatinized starch; stearic acid or magnesium stearate; and croscarmellose sodium, and an acetaminophen layer, wherein the acetaminophen layer comprises acetaminophen; colloidal silicon dioxide; starch and/or partially pregelatinized starch; stearic acid or magnesium stearate, and croscarmellose sodium.

It was unexpectedly observed that a combination tablet containing the roller-compacted granules comprising naproxen sodium, as described herein, and acetaminophen in a bilayer tablet configuration demonstrated a shorter disintegration time than tablets containing naproxen sodium alone, either in the form of the commercially available naproxen sodium tablet prepared by wet granulation or in the form of the naproxen sodium tablets prepared by dry granulation (roller-compaction methods) as described herein.

Without being bound by theory, the shorter disintegration time observed for the bilayer naproxen sodium tablets containing naproxen sodium granules and acetaminophen in separate layers, as compared to the individual active naproxen sodium tablets, was attributed to both the complementary disintegration mechanisms of naproxen sodium and acetaminophen and the confinement of the naproxen sodium to a single half-layer. In contrast to the erosion-based disintegration of naproxen sodium, the disintegration of acetaminophen is directly correlated to the quantity of superdisintegrants present. Due to the presence of superdisintegrants in the bilayer tablet, the disintegration of the acetaminophen layer was observed to occur within tens of seconds. The rapid disintegration of the acetaminophen layer led to an increased surface area exposure of the remaining naproxen sodium half-layer to the disintegration medium, which is believed to have enabled more rapid disintegration of the naproxen sodium layer and resulted in the overall short disintegration time of the bilayer tablet.

The following description sets forth exemplary methods, parameters and the like. It should be recognized, however, that such description is not intended as a limitation on the scope of the present disclosure but is instead provided as a description of exemplary embodiments.

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Oral Monolayer and Bilayer Tablets Comprising Roller-Compacted Granules

In one aspect of the present disclosure, provided herein is an oral tablet, comprising roller-compacted granules and extragranular excipients, wherein the roller-compacted tablets comprise an active pharmaceutical ingredient and intragranular excipients. In some embodiments, the active pharmaceutical ingredient is naproxen sodium and the roller-compacted granules comprise naproxen sodium.

In some embodiments, provided herein is a naproxen sodium tablet comprising roller-compacted granules comprising naproxen sodium; mannitol; colloidal silicon dioxide; one or more lubricants, and superdisintegrant. In still further embodiments, provided herein is a naproxen sodium tablet comprising roller-compacted granules comprising naproxen sodium; mannitol; colloidal silicon dioxide; stearic acid; sodium starch glycolate; and magnesium stearate. In some variations, the naproxen sodium tablet has a dissolution profile wherein at least 80% (±5%) naproxen sodium is dissolved at ten minutes and 100% (±5%) naproxen sodium is dissolved at 20 minutes as determined by the USP apparatus-2 (paddle) Dissolution Test in phosphate buffer of pH 7.4.

### Roller-Compacted Granules

As described herein, the present disclosure provides oral tablets prepared by dry granulation using a roller compaction process. Accordingly, the present disclosure provides oral tablets comprising roller-compacted granules.

The naproxen sodium tablets of the present disclosure possess an enhanced dissolution profile as compared to commercial naproxen sodium tablets prepared by wet granulation. Moreover, the enhanced dissolution profile appears to be primarily the result of unique composition of the tablets—that is, the combination of naproxen sodium with choice and quantity of excipients inside (intragranular) and outside (extragranular) of the roller-compacted granules—with minimal influence from process parameters during roller compaction and tableting. The unique composition and structural properties of the roller-compacted granules, including their particle size distribution and porosity, as described herein contribute to a consistent physical profile, including but not limited to the dissolution and disintegration times.

The term “granule” as described herein may be defined as a solid aggregate or an agglomerated particle comprising two or more fine powder materials in a single mass. The term “roller-compacted granule” as used herein should be understood as referring to a granule prepared by roller compaction.

The roller-compacted granules of the present disclosure comprise at least one active pharmaceutical ingredient, such as naproxen sodium, and intragranular excipients. In some embodiments wherein the active ingredient is naproxen sodium, provided herein is a naproxen sodium tablet comprising roller-compacted granules, wherein the roller-compacted granules comprise naproxen sodium.

In some embodiments, the naproxen sodium tablet comprises at least 75% by weight (or % w/w), at least 80% w/w, at least 85% w/w, or at least 90% w/w roller-compacted granules of the total weight of the naproxen sodium tablet. In other embodiments, the naproxen sodium tablet comprises less than or equal to 95% w/w, less than or equal to 92% w/w, or less than or equal to 90% w/w roller-compacted granules of the total weight of the naproxen sodium tablet. In certain embodiments, the naproxen sodium tablet comprises 75-95% w/w, 75-92% w/w, 75-90% w/w, 80-95% w/w, 80-92% w/w, 80-90% w/w, 85-95% w/w, 85-92% w/w, 85-90% w/w, 90-95% w/w, or 90-92% w/w roller-compacted granules of the total weight of the naproxen sodium tablet.

### Active Pharmaceutical Ingredient

As noted above, the present disclosure provides an oral tablet containing roller-compacted granules, wherein the roller-compacted granules comprise at least one active pharmaceutical ingredient. In some embodiments, provided herein are oral tablets comprising roller-compacted granules comprising naproxen sodium as an active pharmaceutical ingredient. It should be recognized that oral tablets of the present disclosure comprising naproxen sodium may also be referred to as “naproxen sodium tablets”. “Naproxen sodium tablets” may be further characterized as monolayer naproxen sodium tablets or bilayer naproxen sodium tablets.

Naproxen sodium is an active compound in the class of non-steroidal anti-inflammatory drugs (NSAIDs), which are widely used to treat inflammation-related disorders. Naproxen sodium possesses further antipyretic and analgesic properties in addition to its anti-inflammatory effects, and is used to treat various ailments including but not limited to minor pain of arthritis, menstrual cramps, muscular aches, backache, headache, toothache, and the common cold.

In some embodiments, the naproxen sodium tablet comprises at least 50 mg or at least 100 mg naproxen sodium. In other embodiments, the naproxen sodium tablet comprises less than or equal to 300 mg, less than or equal to 250 mg, less than or equal to 200 mg, or less than or equal to 150 mg naproxen sodium. In some embodiments, naproxen sodium tablet comprises between 50 mg and 300 mg, between 50 mg and 250 mg, between 50 mg and 200 mg, between 50 mg and 150 mg, between 100 mg and 300 mg, between 100 mg and 250 mg, between 100 mg and 200 mg, between 150 mg and 300 mg, between 150 mg and 250 mg, between 150 mg and 200 mg, between 200 mg and 300 mg, between 200 mg and 250 mg, or between 250 mg and 300 mg. In certain embodiments, the naproxen sodium tablet comprises 100 mg, 110 mg, 150 mg, 200 mg, 220 mg, 250 mg, or 300 mg naproxen sodium. In certain other embodiments, the naproxen sodium tablet comprises 220 mg naproxen sodium.

In some embodiments, the naproxen sodium tablets described herein comprise at least 40% w/w, at least 50% w/w, at least 60% w/w, or at least 70% w/w naproxen sodium of the total weight of the naproxen sodium tablets. In other embodiments, the naproxen sodium tablets described herein comprise less than or equal to 95% w/w, less than or equal to 90% w/w, less than or equal to 85% w/w, or less than or equal to 80% w/w naproxen sodium of the total weight of the naproxen sodium tablets. In certain embodiments, the naproxen sodium tablets comprise 40-95% w/w, 40-90% w/w, 40-85% w/w, 40-80% w/w%, 50-95% w/w, 50-90% w/w, 50-85% w/w, 50-80% w/w%, 60-95% w/w, 60-90% w/w, 60-85% w/w, 60-80% w/w%, 70-95% w/w, 70-90% w/w, 70-85% w/w, or 70-80% w/w naproxen sodium of the total weight of the naproxen sodium tablets.

In other embodiments, the roller-compacted granules comprise at least 50% w/w, at least 60% w/w, or at least 70% w/w naproxen sodium of the total weight of the roller-compacted granules. In some embodiments, the roller-compacted granules comprise less than or equal to 90% w/w, less than or equal to 80% w/w, or less than or equal to 75% w/w naproxen sodium of the total weight of the roller-compacted granules. In certain embodiments, the roller-compacted granules comprise 50-90% w/w, 50-80% w/w, 50-75% w/w, 60-90% w/w, 60-80% w/w, 70-90% w/w, 70-80% w/w, or 70-75% w/w naproxen sodium of the total weight of the roller-compacted granules.

It should be acknowledged, however, that the dry granulation methods of the present disclosure and compatible excipients therefor may also be suitable for preparing oral tablets comprising other drugs similar to naproxen sodium in pain relieving effect, mechanism of action, chemical structure, physicochemical properties, or any combinations thereof, in lieu of naproxen sodium as the primary active ingredient. Alternatively, it should be recognized that the oral tablets and dry granulation methods, of the present disclosure may be suitable for pharmaceutical combinations comprising multiple active pharmaceutical ingredients, in which naproxen sodium may be one such ingredient. In some embodiments, the oral tablets comprising roller-compacted granules comprising naproxen sodium may comprise one or more additional active pharmaceutical ingredients. In certain embodiments, the oral tablets comprise a single layer containing the roller-compacted granules comprising naproxen sodium and one or more additional active pharmaceutical ingredients. Suitable additional active ingredients may include but are not limited to active pharmaceutical ingredients indicated for the treatment of pain, fever, and/or cold and flu, such as acetaminophen, phenylephrine, pseudoephedrine, doxylamine, dextromethorphan, and/or guaifenesin, or any pharmaceutically acceptable salt thereof.

### Intragranular Excipients

In addition to the active pharmaceutical ingredient, the roller-compacted granules of the oral tablets described herein may further comprise excipients, such as bulking agents, lubricants, distintegrants, superdisintegrants, etc., to provide any desired physical characteristics, such as for downstream manufacture and usage. Such excipients contained within the roller-compacted granules may be referred as intragranular excipients. In some embodiments, the naproxen sodium tablets of the present disclosure having roller-compacted granules further comprise intragranular excipients. As described above, the combination of naproxen sodium with the intragranular excipients allows the formation of porous structure within the resulting granules comprising naproxen sodium described herein and confer enhanced dissolution properties to the resulting naproxen sodium tablets.

In some embodiments, the roller-compacted granules comprising naproxen sodium comprise mannitol. As described another way, in some embodiments, the naproxen sodium tablet comprising roller-compacted granules comprises mannitol as an intragranular excipient. Mannitol is a water-soluble sugar alcohol, used variously as a diluent, bulking agent and disintegrant in a range of types of formulations. Mannitol is also known by various commercial tradenames, including but not limited to Ludiflash®, Mannogem®, and Pearlitol®. In some embodiments, the mannitol is spray-dried mannitol. In some embodiments of the foregoing, the mannitol has an average particle size of at least 50 μm, at least 75 μm, at least 100 μm, at least 125 μm or at least 150 μm. In other embodiments, the mannitol has an average particle size of less than or equal to 300 μm, less than or equal to 275 μm, less than or equal to 250 μm, less than or equal to 225 μm, or less than or equal to 200 μm. In certain embodiments, the mannitol has an average particle size of between 50 μm and 300 μm, between 50 μm and 250 μm, between 50 μm and 200 μm, between 100 μm and 300 μm, between 100 μm and 250 μm, between 100 μm and 200 μm, between 150 μm and 300 μm, between 150 μm and 250 μm, or between 50 μm and 200 μm.

In some embodiments, the roller-compacted granules comprise at least 5% w/w, at least 10% w/w, or at least 15% w/w mannitol of the total weight of the roller-compacted granules. In other embodiments, the roller-compacted granules comprise less than or equal to 30% w/w, less than or equal to 25% w/w, or less than or equal to 20% w/w mannitol of the total weight of the roller-compacted granules. In certain embodiments, the roller-compacted granules comprise 5-30% w/w, 5-25% w/w, 5-20% w/w, 10-30% w/w, 10-25% w/w, 10-20% w/w, 15-30%, 15-25%, or 15-20% w/w mannitol of the total weight of the roller-compacted granules.

It should be recognized that the intragranular mannitol as described above may be substituted or combined with other suitable disintegrants, such as polyols. In some embodiments, the roller-compacted granules comprises one or more polyols. Polyols as utilized in the tablets provided herein may include but are limited to sugar alcohols, such as sorbitol, erythritol, xylitol, mannitol, and lactitol. In certain embodiments, one or more polyols are one or more sugar alcohols. In other embodiments, the roller-compacted granules comprise mannitol, sorbitol, lactitol, or xylitol, or a combination thereof.

In some embodiments, the roller-compacted granules comprise at least 5% w/w, at least 10% w/w, or at least 15% w/w polyol of the total weight of the roller-compacted granules. In other embodiments, the roller-compacted granules comprise less than or equal to 30% w/w, less than or equal to 25% w/w, or less than or equal to 20% w/w polyol of the total weight of the roller-compacted granules. In certain embodiments, the roller-compacted granules comprise 5-30% w/w, 5-25% w/w, 5-20% w/w, 10-30% w/w, 10-25% w/w, 10-20% w/w, 15-30%, 15-25%, or 15-20% w/w polyol of the total weight of the roller-compacted granules.

In some embodiments, the roller-compacted granules comprise colloidal silicon dioxide. In some embodiments, the naproxen sodium tablet comprising roller-compacted granules comprises colloidal silicon dioxide as an intragranular excipient. Colloidal silicon dioxide, also sold as Cab-O-Sil , is a glidant, which can augment the flowability and reduce friction of powder mixtures in the manufacturing process. In some embodiments, the roller-compacted granules comprise at least 0.1% w/w, at least 0.5% w/w, or at least 1% w/w colloidal silicon dioxide of the total weight of the roller-compacted granules. In other embodiments, the roller-compacted granules comprise less than or equal to 10% w/w, less than or equal to 5% w/w, or less than or equal to 2% w/w colloidal silicon dioxide of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w colloidal silicon dioxide of the total weight of the roller-compacted granules.

In some embodiments, the roller-compacted granules comprise one or more lubricants. In some embodiments of the foregoing, the roller-compacted granules comprise stearic acid. As described another way, in some embodiments, the naproxen sodium tablet comprising roller-compacted granules comprises stearic acid or as an intragranular excipient. Stearic acid is both a lubricant and a solubilizing agent, which can help to achieve the desired flowability of powder mixtures during manufacture as well as dissolution profile in actual usage of the tablet. In some embodiments, the roller-compacted granules comprise at least 0.1% w/w, at least 0.5% w/w, or at least 1% w/w stearic acid of the total weight of the roller-compacted granules. In other embodiments, the roller-compacted granules comprise less than or equal to 10% w/w, less than or equal to 5% w/w, or less than or equal to 2% w/w stearic acid of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w stearic acid of the total weight of the roller-compacted granules.

In other embodiments, sodium stearyl fumarate may be utilized in lieu of stearic acid as a lubricant. In some embodiments, the roller-compacted granules comprise sodium stearyl fumarate. As described another way, in some embodiments, the naproxen sodium tablet comprising roller-compacted granules comprises sodium stearyl fumarate as an intragranular excipient. Similar to stearic acid, sodium stearyl fumarate is a lubricant, which can be used to modulate flowability of the granules. In some embodiments, the roller-compacted granules comprise at least 0.1% w/w, at least 0.5% w/w, or at least 1% w/w sodium stearyl fumarate of the total weight of the roller-compacted granules. In other embodiments, the roller-compacted granules comprise less than or equal to 10% w/w, less than or equal to 5% w/w, or less than or equal to 2% w/w sodium stearyl fumarate of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w sodium stearyl fumarate of the total weight of the roller-compacted granules.

In still other embodiments, the roller-compacted granules comprise magnesium stearate . Similar to stearic acid and sodium stearyl fumarate, magnesium stearate also behaves a lubricant. In some embodiments, the roller-compacted granules comprise at least 0.1% w/w, at least 0.5% w/w, or at least 1% w/w magnesium stearate of the total weight of the roller-compacted granules. In other embodiments, the roller-compacted granules comprise less than or equal to 10% w/w, less than or equal to 5% w/w, or less than or equal to 2% w/w magnesium stearate of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w magnesium stearate of the total weight of the roller-compacted granules.

In still other embodiments, stearic acid, sodium stearyl fumarate, and magnesium stearate may be used in combination as lubricants.

In some embodiments, the roller-compacted granules comprise stearic acid and sodium stearyl fumarate. In other embodiments, the naproxen sodium tablet comprises stearic acid and sodium stearyl fumarate as intragranular excipients. In some embodiments wherein stearic acid and sodium stearyl fumarate as both utilized as intragranular excipients, the roller-compacted granules comprise at least 0.1% w/w, at least 0.5% w/w, or at least 1% w/w a combination of stearic acid and sodium stearyl fumarate of the total weight of the roller-compacted granules. In other embodiments, the roller-compacted granules comprise less than or equal to 10% w/w, less than or equal to 5% w/w, or less than or equal to 2% w/w a combination of stearic acid and sodium stearyl fumarate of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w a combination of stearic acid and sodium stearyl fumarate of the total weight of the roller-compacted granules.

In some embodiments, the roller-compacted granules comprise stearic acid and magnesium stearate. In other embodiments, the naproxen sodium tablet comprises stearic acid and magnesium stearate as intragranular excipients. In some embodiments wherein stearic acid and magnesium stearate as both utilized as intragranular excipients, the roller-compacted granules comprise at least 0.1% w/w, at least 0.5% w/w, or at least 1% w/w a combination of stearic acid and magnesium stearate of the total weight of the roller-compacted granules. In other embodiments, the roller-compacted granules comprise less than or equal to 10% w/w, less than or equal to 5% w/w,or less than or equal to 2% w/w a combination of stearic acid and magnesium stearate of the total weight of the roller-compacted granules. In certain embodiments, the roller-compacted granules comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w a combination of stearic acid and magnesium stearate of the total weight of the roller-compacted granules.

In some embodiments, the roller-compacted granules comprise one or more superdisintegrants. In some embodiments of the foregoing, the roller-compacted granules comprise sodium starch glycolate. In an alternative description, in some embodiments, the naproxen sodium tablet comprising roller-compacted granules comprises sodium starch glycolate as an intragranular excipient. Sodium starch glycolate—the sodium salt of carboxymethyl ether, which is also commercially known as Explotab® or Primogel®—is commonly employed in pharmaceutical dosage forms as superdisintegrant. As a highly hygroscopic, porous material, sodium starch glycolate facilitates the conduction and penetration of water throughout a dosage form, thereby reducing dissolution and disintegration time. In some embodiments, the roller-compacted granules comprise at least 0.1% w/w, at least 0.5% w/w, or at least 1% w/w sodium starch glycolate of the total weight of the roller-compacted granules. In other embodiments, the roller-compacted granules comprise less than or equal to 10% w/w, less than or equal to 5% w/w, less than or equal to 3% w/w sodium starch glycolate of the total weight of the roller-compacted granules. In certain embodiments, the roller-compacted granules comprise 0.1-10% w/w, 0.1-5% w/w, 0.1-3% w/w, 0.5-10% w/w, 0.5-5% w/w, 0.5-3% w/w, 1-10% w/w, 1-5% w/w, or 1-3% w/w sodium starch glycolate of the total weight of the roller-compacted granules.

In other embodiments, the roller-compacted granules comprise at least 5% w/w, at least 10% w/w, at least 15% w/w, or at least 20% w/w intragranular excipients of the total weight of the roller-compacted granules. In other embodiments, the roller-compacted granules comprise less than or equal to 40% w/w, less than or equal to 35% w/w, less than or equal to 30% w/w, or less than or equal to 25% w/w intragranular excipients of the total weight of the roller-compacted granules. In certain embodiments, the roller-compacted granules comprise 5-40% w/w, 5-35% w/w, 5-30% w/w, 5-25% w/w, 10-40% w/w, 10-35% w/w, 10-30% w/w, 10-25% w/w, 15-40% w/w, 15-35% w/w, 15-30% w/w, 15-25% w/w, 20-40% w/w, 20-35% w/w, 20-30% w/w, or 20-25% w/w intragranular excipients of the total weight of the roller-compacted granules.

In some embodiments, the roller-compacted granules comprise naproxen sodium, mannitol, colloidal silicon dioxide, stearic acid, and sodium starch glycolate. In other embodiments, the roller-compacted granules comprise naproxen sodium, mannitol, colloidal silicon dioxide, sodium stearyl fumarate, and sodium starch glycolate. In some embodiments, the roller-compacted granules comprise naproxen sodium, mannitol, colloidal silicon dioxide, magnesium stearate, and sodium starch glycolate. In yet other embodiments, the roller-compacted granules comprise naproxen sodium, mannitol, colloidal silicon dioxide, stearic acid, sodium stearyl fumarate, and sodium starch glycolate. In some embodiments, the roller-compacted granules comprise naproxen sodium, mannitol, colloidal silicon dioxide, stearic acid, magnesium stearate, and sodium starch glycolate. In still further embodiments, the naproxen sodium tablet comprises a combination of mannitol, colloidal silicon dioxide, stearic acid, and sodium starch glycolate as intragranular excipients. In some embodiments, the naproxen sodium tablet comprises a combination of mannitol, colloidal silicon dioxide, sodium stearyl fumarate, and sodium starch glycolate as intragranular excipients. In other embodiments, the naproxen sodium tablet comprises a combination of mannitol, colloidal silicon dioxide, stearic acid, sodium stearyl fumarate, and sodium starch glycolate as intragranular excipients. In other embodiments, the naproxen sodium tablet comprises a combination of mannitol, colloidal silicon dioxide, stearic acid, magnesium stearate, and sodium starch glycolate as intragranular excipients.

### Extragranular Excipients

As described herein, the oral tablets comprising roller-compacted granules may comprise the active pharmaceutical ingredient(s) along with intragranular excipients within the granules and extragranular excipients outside of the granules. Additional excipients (extragranular excipients) may be added to the roller-compacted granules to help to bind the granules together, provide volume/body to granules for compression, and confer structural stability to the final tablets. Suitable extragranular excipients may include but are not limited excipients such as binders, lubricants, distintegrants, superdisintegrants, etc. In some embodiments, the naproxen sodium tablets of the present disclosure having roller-compacted granules further comprise extragranular excipients.

In some embodiments, the naproxen sodium tablet comprises mannitol as an extragranular excipient. As described above, mannitol is a sugar alcohol that may serve in a variety of excipient roles including, for example, diluent, bulking agent, and disintegrant. In some embodiments, the naproxen sodium tablet comprises at least 0.5% w/w, at least 0.75% w/w, at least 1% w/w, at least 2% w/w, or at least 3% w/w extragranular mannitol of the total weight of the naproxen sodium tablet. In other embodiments, the naproxen sodium tablet comprises less than or equal to 10% w/w, less than or equal to 7% w/w, or less than or equal to 5% w/w extragranular mannitol of the total weight of the naproxen sodium tablet. In certain embodiments the naproxen sodium tablet comprises 0.5-10% w/w, 0.5-7%, 0.5-5%, 0.75-10% w/w, 0.75-7%, 0.75-5%, 1-10% w/w, 1-7% w/w, 1-5% w/w, 2-10% w/w, 2-7% w/w, 2-5% w/w, 3-10% w/w, 3-7% w/w, or 3-5% w/w extragranular mannitol of the total weight of the naproxen sodium tablet.

In some embodiments, the certain properties or types of mannitol may be particularly useful for extragranular mannitol as described herein. In some embodiments, the mannitol is spray-dried mannitol. In some embodiments of the foregoing, the mannitol has an average particle size of at least 50 μm, at least 75 μm, at least 100 μm, at least 125 μm or at least 150 μm. In other embodiments, the mannitol has an average particle size of less than or equal to 300 μm, less than or equal to 275 μm, less than or equal to 250 μm, less than or equal to 225 μm, or less than or equal to 200 μm. In certain embodiments, the mannitol has an average particle size of between 50 μm and 300 μm, between 50 μm and 250 μm, between 50 μm and 200 μm, between 100 μm and 300 μm, between 100 μm and 250 μm, between 100 μm and 200 μm, between 150 μm and 300 μm, between 150 μm and 250 μm, or between 50 μm and 200 μm.

As with the intragranular mannitol above, it should be further recognized that the extragranular mannitol as described above may also be substituted or combined with other suitable disintegrants, such as polyols. In some embodiments, the naproxen sodium tablet comprises one or more polyols as an extragranular excipient. Polyols as utilized in the tablets provided herein may include but are limited to sugar alcohols, such as sorbitol, erythritol, xylitol, mannitol, and lactitol. In certain embodiments, one or more polyols are one or more sugar alcohols. In other embodiments, the naproxen sodium tablet comprises mannitol, sorbitol, lactitol, or xylitol, or a combination thereof, as an extragranular excipient.

In some embodiments, the naproxen sodium tablet comprises a polyol as an extragranular excipient. In some embodiments, the naproxen sodium tablet comprises at least 0.5% w/w, at least 0.75% w/w, at least 1% w/w, at least 2% w/w, or at least 3% w/w extragranular polyol of the total weight of the naproxen sodium tablet. In other embodiments, the naproxen sodium tablet comprises less than or equal to 10% w/w, less than or equal to 7% w/w, or less than or equal to 5% w/w extragranular polyol of the total weight of the naproxen sodium tablet. In certain embodiments the naproxen sodium tablet comprises 0.5-10% w/w, 0.5-7%, 0.5-5%, 0.75-10% w/w, 0.75-7%, 0.75-5%, 1-10% w/w, 1-7% w/w, 1-5% w/w, 2-10% w/w, 2-7% w/w, 2-5% w/w, 3-10% w/w, 3-7% w/w, or 3-5% w/w extragranular polyol of the total weight of the naproxen sodium tablet.

In still further embodiments, the naproxen sodium tablet comprises one or more extragranular superdisintegrants. In some embodiments, the naproxen sodium tablet comprises sodium starch glycolate as an extragranular excipient. As described above, sodium starch glycolate may be utilized as a superdisintegrant in pharmaceutical formulations to facilitate the conduction and penetration of dissolution media throughout the naproxen sodium tablet. In some embodiments, the naproxen sodium tablet comprises at least 0.1% w/w, at least 0.5% w/w, at least 1% w/w, or at least 2% w/w extragranular sodium starch glycolate of the total weight of the naproxen sodium tablet. In other embodiments, the naproxen sodium tablet comprises less than or equal to 10% w/w, 7% w/w, 5% w/w, or 3% w/w extragranular sodium starch glycolate of the total weight of the naproxen sodium tablet. In certain embodiments, the naproxen sodium tablet comprises 0.1-10% w/w, 0.1-7% w/w, 0.1-5% w/w, 0.1-3% w/w, 0.5-10% w/w, 0.5-7% w/w, 0.5-5% w/w, 0.5-3% w/w, 1-10% w/w, 1-7% w/w, 1-5% w/w, 1-3% w/w, 2-10% w/w, 2-7% w/w, 2-5% w/w, or 2-3% w/w extragranular sodium starch glycolate of the total weight of the naproxen sodium tablet.

In yet other embodiments, the naproxen sodium tablet comprises one or more extragranular lubricants. In some embodiments, the naproxen sodium tablet comprises magnesium stearate as an extragranular excipient. Magnesium stearate is an excipient used in various dosage forms as a lubricant to reduce sticking of powder material to processing equipment and facilitate the discharge of tablets from tablet presses. In some embodiments, the naproxen sodium tablet comprises at least 0.1% w/w, at least 0.5% w/w, or at least 1% w/w extragranular magnesium stearate of the total weight of the naproxen sodium tablet. In other embodiments, the naproxen sodium tablet comprises less than or equal to 10% w/w, 7% w/w, 5% w/w, or 2% w/w extragranular magnesium stearate of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w extragranular magnesium stearate of the total weight of the naproxen sodium tablet.

In still other embodiments, the naproxen sodium tablet comprises colloidal silicon dioxide as an extragranular excipient. In some embodiments, the naproxen sodium tablet comprises at least 0.1% w/w, at least 0.5% w/w, or at least 1% w/w extragranular colloidal silicon dioxide of the total weight of the naproxen sodium tablet. In other embodiments, the naproxen sodium tablet comprises less than or equal to 10% w/w, 7% w/w, 5% w/w, or 2% w/w extragranular colloidal silicon dioxide of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w extragranular colloidal silicon dioxide of the total weight of the naproxen sodium tablet.

In some embodiments, the naproxen sodium tablet comprises stearic acid as an extragranular excipient. In some embodiments, the naproxen sodium tablet comprises at least 0.1% w/w, at least 0.5% w/w, or at least 1% w/w extragranular stearic acid of the total weight of the naproxen sodium tablet. In other embodiments, the naproxen sodium tablet comprises less than or equal to 10% w/w, 7% w/w, 5% w/w, or 2% w/w extragranular stearic acid of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w extragranular stearic acid of the total weight of the naproxen sodium tablet.

In other embodiments, the naproxen sodium tablet comprises sodium stearyl fumarate as an extragranular excipient. In some embodiments, the naproxen sodium tablet comprises at least 0.1% w/w, at least 0.5% w/w, or at least 1% w/w extragranular sodium stearyl fumarate of the total weight of the naproxen sodium tablet. In other embodiments, the naproxen sodium tablet comprises less than or equal to 10% w/w, 7% w/w, 5% w/w, or 2% w/w extragranular sodium stearyl fumarate of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w extragranular sodium stearyl fumarate of the total weight of the naproxen sodium tablet. In still further embodiments, the naproxen sodium tablet comprises a combination of stearic acid and sodium stearyl fumarate as an extragranular excipient. In some embodiments, the naproxen sodium tablet comprises at least 0.1% w/w, at least 0.5% w/w, or at least 1% w/w a combination of stearic acid and sodium stearyl fumarate as extragranular excipients of the total weight of the naproxen sodium tablet. In other embodiments, the naproxen sodium tablet comprises less than or equal to 10% w/w, 7% w/w, 5% w/w, or 2% w/w a combination of stearic acid and sodium stearyl fumarate as extragranular excipients of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w a combination of stearic acid and sodium stearyl fumarate as extragranular excipients of the total weight of the naproxen sodium tablet.

In some embodiments, the naproxen sodium tablet comprises as extragranular excipients mannitol, sodium starch glycolate, magnesium stearate, colloidal silicon dioxide, stearic acid, sodium stearyl fumarate, or any combinations thereof. In some embodiments, the naproxen sodium tablet comprises mannitol, sodium starch glycolate, magnesium stearate, and optionally colloidal silicon dioxide as extragranular excipients. In still further embodiments, the naproxen sodium tablet comprises mannitol, sodium starch glycolate and magnesium stearate as extragranular excipients. In yet other embodiments, the naproxen sodium tablet comprises mannitol, sodium starch glycolate, magnesium stearate, and colloidal silicon dioxide as extragranular excipients.

In still other embodiments, embodiments, the oral tablets comprising roller-compacted granules comprising naproxen sodium may comprise one or more additional active pharmaceutical ingredients external to the roller-compacted granules. In certain embodiments, the oral tablets comprise a single layer containing the roller-compacted granules comprising naproxen sodium, and one or more additional extragranular active pharmaceutical ingredients. Suitable additional extragranular active ingredients may include but are not limited to active pharmaceutical ingredients indicated for the treatment of pain, fever, and/or cold and flu, such as acetaminophen, phenylephrine, pseudoephedrine, doxylamine, dextromethorphan, and/or guaifenesin, or any pharmaceutically acceptable salt thereof (e.g., pseudoephedrine hydrochloride or pseudoephedrine sulfate).

### Intragranular and Extragranular Excipients

As described herein, the naproxen sodium tablet may comprise one or more excipients as both intragranular and extragranular excipients. Excipients of the present disclosure which may be suitable for use as an intragranular excipient, an extragranular excipient or both, may include but are not limited to mannitol, colloidal silicon dioxide, sodium starch glycolate.

For example, in some embodiments, the naproxen sodium tablet comprises mannitol as both an intragranular excipient and extragranular excipient. In some embodiments wherein the naproxen sodium tablet comprises intragranular mannitol and extragranular mannitol, the naproxen sodium tablet comprises at least 5% w/w, at least 10% w/w, or at least 15% w/w mannitol of the total weight of the naproxen sodium tablet. In other embodiments, naproxen sodium tablet comprises less than or equal to 40% w/w, less than or equal to 30% w/w, or less than or equal to 20% w/w mannitol of the total weight of the naproxen sodium tablet. In certain embodiments, the naproxen sodium tablet comprises 5-40% w/w, 5-30% w/w, 5-20% w/w, 10-40% w/w, 10-30% w/w, 10-20% w/w, 15-40% w/w, 15-30% w/w, or 15-20% w/w mannitol of the total weight of the naproxen sodium tablet.

In still further embodiments wherein the tablet comprises one or more polyols (such as sorbitol, erythritol, xylitol, mannitol, and lactitol) as intragranular and extragranular excipients, the naproxen sodium tablet comprises at least 5% w/w, at least 10% w/w, or at least 15% w/w one or more polyols of the total weight of the naproxen sodium tablet. In other embodiments, naproxen sodium tablet comprises less than or equal to 40% w/w, less than or equal to 30% w/w, or less than or equal to 20% w/w one or more polyols of the total weight of the naproxen sodium tablet. In certain embodiments, the naproxen sodium tablet comprises 5-40% w/w, 5-30% w/w, 5-20% w/w, 10-40% w/w, 10-30% w/w, 10-20% w/w, 15-40% w/w, 15-30% w/w, or 15-20% w/w one or more polyols of the total weight of the naproxen sodium tablet.

In some embodiments wherein the naproxen sodium tablet comprises sodium starch glycolate as both an intragranular and extragranular excipient, the naproxen sodium tablet comprises at least about 1% w/w, at least about 2% w/w, at least about 3% w/w, or at least about 4% w/w sodium starch glycolate. In other embodiments wherein the naproxen sodium tablet comprises intragranular and extragranular sodium starch glycolate, the naproxen sodium tablet comprises less than or equal to 10% w/w, less than or equal to 9% w/w, less than or equal to 8% w/w, less than or equal to 7% w/w, less than or equal to 6% w/w, or less than or equal to 5% w/w sodium starch glycolate. In certain embodiments wherein the naproxen sodium tablet comprises sodium starch glycolate as both an intragranular and extragranular excipient, the naproxen sodium tablet comprises 1-10% w/w, 1-7% w/w, 1-5% w/w, 1-3% w/w, 2-10% w/w, 2-8% w/w, 2-6% w/w, 2-4% w/w, 3-10% w/w, 3-9% w/w, 3-7% w/w, 3-5% w/w, 4-10% w/w, 4-8% w/w, 4-6% w/w, or 4-5% w/w sodium starch glycolate.

In yet further embodiments, the naproxen sodium tablet comprises colloidal silicon dioxide as both an intragranular excipient and an extragranular excipient. In some embodiments, the naproxen sodium tablet comprises at least 0.1% w/w, at least 0.5% w/w, or at least 1% w/w colloidal silicon dioxide of the total weight of the naproxen sodium tablet. In other embodiments, the naproxen sodium tablet comprises less than or equal to 10% w/w, 7% w/w, 5% w/w, or 2% w/w colloidal silicon dioxide of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w colloidal silicon dioxide of the total weight of the naproxen sodium tablet.

In some embodiments, the naproxen sodium tablet comprises stearic acid as both an intragranular excipient and an extragranular excipient. In some embodiments, the naproxen sodium tablet comprises at least 0.1% w/w, at least 0.5% w/w, or at least 1% w/w stearic acid of the total weight of the naproxen sodium tablet. In other embodiments, the naproxen sodium tablet comprises less than or equal to 10% w/w, 7% w/w, 5% w/w, or 2% w/w stearic acid of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w stearic acid of the total weight of the naproxen sodium tablet.

In some embodiments, the naproxen sodium tablet comprises sodium stearyl fumarate as both an intragranular excipient and an extragranular excipient. In some embodiments, the naproxen sodium tablet comprises at least 0.1% w/w, at least 0.5% w/w, or at least 1% w/w sodium stearyl fumarate of the total weight of the naproxen sodium tablet. In other embodiments, the naproxen sodium tablet comprises less than or equal to 10% w/w, 7% w/w, 5% w/w, or 2% w/w sodium stearyl fumarate of the total weight of the naproxen sodium tablet. In certain embodiments, the naproxen sodium tablet comprises 0.1-10% w/w, 0.1-7% w/w, 0.1-5% w/w, 0.1-2% w/w, 0.5-10% w/w, 0.5-7% w/w, 0.5-5% w/w, 0.5-2% w/w, 1-10% w/w, 1-7% w/w, 1-5% w/w, or 1-2% w/w sodium stearyl fumarate of the total weight of the naproxen sodium tablet.

It should be noted that the dry granulation (roller compaction) methods of the present disclosure differ from the wet granulation methods more commonly used in the production of naproxen sodium tablets in that a liquid granulation fluid is not utilized. As such, in some embodiments, the naproxen sodium tablet does not contain water and/or ethanol.

In other embodiments, the naproxen sodium tablets of the present disclosure do not contain certain ingredients as intragranular or extragranular excipients, which may be utilized to enhance intragranular binding in wet granulation. For example, in some embodiments, the naproxen sodium tablets described herein do not contain microcrystalline cellulose (MCC), hydroxypropyl methylcellulose (HPMC) or other cellulose derivatives. In some embodiments, the naproxen sodium tablet does not contain polyvinylpyrrolidone (povidone) or derivatives such as cross-linked polyvinylpyrrolidone (crospovidone) or polyvinylpyrrolidone-vinyl acetate copolymer (copovidone). In other embodiments, the naproxen sodium tablet does not contain croscarmellose sodium. In some embodiments, the naproxen sodium tablet does not contain polyethylene glycol. In yet other embodiments, the naproxen sodium tablet does not contain cornstarch or talc.

### Coating

In some embodiments, the naproxen sodium tablet further comprises a film coating. In some variations, the film coating comprises poly(vinylalcohol). In certain embodiments, the film coating is an immediate release coating.

In other embodiments, the film coating further comprises a colorant, a flavorant, or a combination thereof.

### Tablet Dissolution, Disintegration and Other Properties

By virtue of the careful selection of excipients combined with roller compaction, the naproxen sodium tablets of the present disclosure have drug release profile in which the tablet disintegrates in a relatively short amount of time and the active pharmaceutical ingredient dissolves in solution at a faster rate than existing naproxen sodium tablets.

Naproxen and naproxen sodium are insoluble in acidic media, including at gastric pH. The in vivo absorption of naproxen sodium occurs principally within the small intestine. The drug release profile of naproxen tablets, including the naproxen sodium tablets may thus be characterized under conditions that may be better reflective of the environment provided by the various sections of the small intestine, e.g., at pH 7.4 and/or pH 5.8.

In one aspect, provided herein is a naproxen sodium tablet, comprising roller-compacted granules comprising naproxen sodium, wherein the naproxen sodium tablet has an enhanced dissolution profile. Dissolution is a measure of the amount of active ingredient(s) released from a given dosage form into solution over time under standardized conditions. The U.S. Pharmacopeia provides a standardized protocol for evaluating dissolution of naproxen sodium tablets (USP34-NF29, Chapter <711> Dissolution, Stage 6 Harmonization Bulletin dated Dec. 1, 2011; and Naproxen Sodium monograph USP41-NF36, Interim Revision Announcement dated May 1, 2018, 0.1 M phosphate buffer of pH 7.4, 900mL, 37° C.±0.5° C., apparatus 2, 50 rpm, 45 min). As described herein, the term “about” as used in reference to the percentage of active pharmaceutical ingredient (naproxen sodium) dissolved should be understood to encompass variation of ±5%.

In some embodiments, the naproxen sodium tablet has a dissolution profile wherein at least about 70%, at least about 75%, or at least about 80% naproxen sodium is dissolved at 10 minutes as determined by the USP apparatus-2 (paddle) Dissolution Test in phosphate buffer of pH 7.4 at 37° C.±0.5° C. In other embodiments, the naproxen sodium tablet has a dissolution profile wherein less than or equal to about 95%, less than or equal to about 90%, or less than or equal to about 85% naproxen sodium is dissolved at 10 minutes as determined by the USP apparatus-2 (paddle) Dissolution Test in phosphate buffer of pH 7.4 at 37° C.±0.5° C. In certain embodiments, the naproxen sodium tablet has a dissolution profile wherein between 70% and 95%, between 70% and 90%, between 70% and 85%, between 75% and 95%, between 75% and 90%, between 75% and 85%, between 80% and 95%, between 80% and 90%, or between 80% and 85% naproxen sodium is dissolved at 10 minutes as determined by the USP apparatus-2 (paddle) Dissolution Test in phosphate buffer of pH 7.4 at 37° C.±0.5° C.

In other embodiments, the naproxen sodium tablet has a dissolution profile wherein at least about 85%, at least about 90%, at least about 95%, at least about 97%, or at least about 99% naproxen sodium is dissolved at 20 minutes as determined by the USP apparatus-2 (paddle) Dissolution Test in phosphate buffer of pH 7.4 at 37° C.±0.5° C.

In still further embodiments, the naproxen sodium tablet has a dissolution profile wherein at least 70%, at least 75%, or at least about 80% naproxen sodium is dissolved at 10 minutes, and at least about 85%, at least about 90%, at least about 95%, at least about 97% or at least about 99% naproxen sodium is dissolved at 20 minutes as determined by the USP apparatus-2 (paddle) Dissolution Test in phosphate buffer of pH 7.4 at 37° C.±0.5° C. In certain embodiments, the naproxen sodium tablet has a dissolution profile wherein at least about 80% naproxen sodium is dissolved at 10 minutes and about 100% naproxen sodium is dissolved at 20 minutes as determined by the USP apparatus-2 (paddle) Dissolution Test in phosphate buffer of pH 7.4 at 37° C.±0.5° C.

Surprisingly, it was also observed that the naproxen sodium tablets of the present disclosure exhibited a similar dissolution profile when evaluated at pH 5.4, as observed at pH 7.4. The observation of the similar dissolution profile of the naproxen sodium tablets described herein was contrasted by the dissolution of naproxen sodium tablets prepared by traditional wet granulation. To assess the dissolution profile of the naproxen sodium tablets under acidic conditions, the USP standardized dissolution protocol for naproxen sodium tablets was adapted, using phosphate buffer at pH 5.8 instead of slightly basic pH 7.4 with all other parameters held identical (900mL, apparatus 2, 50 rpm, 45 min, at 37° C.±0.5° C.).

In some embodiments, the naproxen sodium tablet has a dissolution profile wherein at least about 70%, at least about 75%, or at least about 80% naproxen sodium is dissolved at 10 minutes as determined by the USP apparatus-2 (paddle) Dissolution Test in phosphate buffer of pH 5.8 at 37° C.±0.5° C. In other embodiments, the naproxen sodium tablet has a dissolution profile wherein less than or equal to about 95%, less than or equal to about 90%, or less than or equal to about 85% naproxen sodium is dissolved at 10 minutes as determined by the USP apparatus-2 (paddle) Dissolution Test in phosphate buffer of pH 5.8 at 37° C.±0.5° C. In certain embodiments, the naproxen sodium tablet has a dissolution profile wherein between 70% and 95%, between 70% and 90%, between 70% and 85%, between 75% and 95%, between 75% and 90%, between 75% and 85%, between 80% and 95%, between 80% and 90%, or between 80% and 85% naproxen sodium is dissolved at 10 minutes as determined by the USP apparatus-2 (paddle) Dissolution Test in phosphate buffer of pH 5.8 at 37° C.±0.5° C.

In other embodiments, the naproxen sodium tablet has a dissolution profile wherein at least about 85%, at least about 90%, at least about 95%, at least about 97%, or at least about 99% naproxen sodium is dissolved at 20 minutes as determined by the USP apparatus-2 (paddle) Dissolution Test in phosphate buffer of pH 5.8 at 37° C.±0.5° C.

In still further embodiments, the naproxen sodium tablet has a dissolution profile wherein at least 70%, at least 75%, or at least about 80% naproxen sodium is dissolved at 10 minutes, and at least about 85%, at least about 90%, at least about 95%, at least about 97% or at least about 99% naproxen sodium is dissolved at 20 minutes as determined by the USP apparatus-2 (paddle) Dissolution Test in phosphate buffer of pH 5.8 at 37° C.±0.5° C. In other embodiments, the naproxen sodium tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer of pH 5.8 at 37° C.±0.5° C.

In other embodiments, the naproxen sodium tablet of the present disclosure has an enhanced disintegration profile. Complete disintegration is defined as a state in which any residue of the unit, except fragments of insoluble coating or capsule shell, remaining on the screen of the test apparatus or adhering to the lower surface of the disk, if used, is a soft mass having no palpably firm core. The disintegration of a tablet can be determined using the protocol as described in the USP-NF (USP43-NF38, Chapter <701> Disintegration, Stage 4 Harmonization Bulletin dated April 26, 2019; uncoated tablet procedure, basket-rack assembly at 37° C.±0.5° C.). Briefly, the protocol involves the submersion of six identical uncoated or plain coated tablets into individual tubes (e.g., basket-rack assembly with disks) containing water or a specified medium for a given active ingredient for a prescribed period of time at a fixed temperature (such as at 37° C.±0.5° C.). The disintegration of the tablets are visually assessed.

The disintegration time of naproxen sodium tablets of the present disclosure may be determined by the USP Disintegration Test in water at 37° C.±0.5° C. using a basket-rack assembly with disks. For example, in some embodiments, the naproxen sodium tablet has a disintegration time of less than about 8 minutes, less than about 7 minutes, less than about 6 minutes, less than about 5 minutes, or less than about 4 minutes as determined by USP Disintegration Test in water at 37° C.±0.5° C. using a basket-rack assembly with disks. In other embodiments, the naproxen sodium tablet has a disintegration time of at least about 1 minute, at least about 2 minutes or at least about 3 minutes as determined by the USP Disintegration Test in water at 37° C.±0.5° C. using a basket-rack assembly with disks. In certain embodiments, the naproxen sodium tablet has a disintegration time of between 1 minute and 8 minutes, between 1 minutes and 7 minutes, between 1 minute and 6 minutes, between 1 minute and 5 minutes, between 1 minute and 4 minutes, between 2 minutes and 8 minutes, between 2 minutes and 7 minutes, between 2 minutes and 6 minutes, between 2 minutes and 5 minutes, between 2 minutes and 4 minutes, between 3 minutes and 8 minutes, between 3 minutes and 7 minutes, between 3 minutes and 6 minutes, between 3 minutes and 5 minutes, between 3 minutes and 4 minutes, as determined by the USP disintegration test in water at 37° C.±0.5° C. using a basket-rack assembly with disks. In other embodiments, the naproxen sodium tablet is not an orally disintegrating tablet.

In addition to their pharmacokinetic properties, the naproxen sodium tablets of the present disclosure may also be characterized by other properties such as physical durability and structural integrity. Physical durability and structural integrity are additional considerations in assessing the commercial viability of pharmaceutical dosage form.

Tablet hardness (or tablet breaking force) is a property that may be used to quantify the structural integrity of a tablet under various conditions to which it might be exposed in storage, transportation, and handling before usage. Hardness may be determined by compression testing methods known in the art, such as the methods described in USP Chapter <1217> Tablet Breaking Force (USP35-NF30 Chapter <1217> Tablet Breaking Force, dated May 1, 2012), and suitable measuring instruments, such as tablet (hardness) testers therein. Hardness is reported as the mechanical force required to cause the tablet to fracture (tablet breaking force). In some embodiments, the naproxen sodium tablet has a hardness of at least 2 kilopond (kp), at least 3 kp, at least 4 kp, at least 5 kp, or at least 6 kp as determined by a tablet tester in accordance with the USP Tablet Breaking Force Test. In other embodiments, the naproxen sodium tablet has a hardness of less than or equal to 18 kp, less than or equal to 17 kp, less than or equal to 16 kp, less than or equal to 15 kp, less than or equal to 14 kp, less than or equal to 13 kp, or less than or equal to 12 kp as determined by a tablet tester in accordance with the USP Tablet Breaking Force Test. In certain embodiments, the naproxen sodium tablet has a hardness between 2 and 18 kp, between 2 and 16 kp, between 2 and 14 kp, between 2 and 12 kp, between 4 and 18 kp, between 4 and 16 kp, between 4 and 14 kp, between 4 and 12 kp, between 6 and 18 kp, between 6 and 16 kp, between 6 and 14 kp, or between 6 and 12 kp as determined by a tablet tester in accordance with the USP Tablet Breaking Force Test.

Friability is another common property that may be used to evaluate the durability of tablets and their tendency to break up into smaller pieces under light pressure or frictional contact. Methods and testing instruments (friability testers) for measuring friability are described in USP Chapter <1216> Tablet Friability (USP35-NF30, Chapter <1216> Friability, dated May 1, 2012). In brief, pre-weighed compressed, uncoated tablets to be evaluated are placed in a rotating drum having an internal diameter of between 283 and 291 mm and a depth between 36 and 40 mm. The drum is made of a transparent synthetic polymer with polished internal surfaces and subject to minimum static build up. The drum is attached to the horizontal axis of a device that rotates at 25±1 revolutions per minute (rpm) and tumbles the enclosed tablets by means of a curved projection (having an inside radius of between 75.5 and 85.5 mm) within the body of the drum that extends from the middle of the drum to the outer wall. The compressed tablets are tumbled in the rotating drum for a fixed number of revolutions, such as for a total of 100 revolutions or 200 revolutions. The tablets are removed from the drum, weighed, and examined for cracks, cleavages or breakages. Friability of a tablet is reported as the percentage of tablet mass lost through chipping.

In some embodiments, the naproxen sodium tablet has a friability of at least 0.1%, at least 0.2%, at least 0.3%, at least 0.4%, or at least 0.5% as determined by the USP Friability Test after 200 revolutions. In other embodiments, the naproxen sodium tablet has a friability of less than or equal to 1%, less than or equal to 0.9%, less than or equal to 0.8%, less than or equal to 0.7%, less than or equal to 0.6%, or less than or equal to 0.5% as determined by the USP Friability Test after 200 revolutions. In certain embodiments, the naproxen sodium tablet has a friability of between 0.1% and 1%, between 0.1% and 0.9%, between 0.1% and 0.7%, between 0.1% and 0.5%, between 0.3% and 1%, between 0.3% and 0.9%, between 0.3% and 0.7%, between 0.3% and 0.5%, between 0.5% and 1%, between 0.5% and 0.9%, or between 0.5% and 0.7% as determined by the USP Friability Test after 200 revolutions.

### Combination Oral Bilayer Tablets Comprising Roller-Compacted Naproxen Sodium Granules

In still yet another aspect, provided herein are combination bilayer oral tablets comprising roller-compacted granules comprising naproxen sodium, and one or more additional active pharmaceutical ingredients. Suitable additional active pharmaceutical ingredients that may be combined with the roller-compacted granules comprising naproxen sodium may include but are not limited to acetaminophen, phenylephrine, pseudoephedrine, doxylamine, dextromethorphan and/or guaifenesin, or any pharmaceutically acceptable salt thereof. In one aspect, provided herein is a bilayer oral tablet comprising roller-compacted granules containing naproxen sodium, and acetaminophen.

As described above, the oral tablets comprising granules comprising naproxen sodium may be combined with one or more other active pharmaceutical ingredients to provide a pharmaceutical combination dosage form, in which naproxen sodium may be one such ingredient. For example, acetaminophen is an antipyretic and analgesic, which may be suitable as a complementary active ingredient to naproxen sodium based upon their respective onsets of action and half-lives.

Surprisingly, it was observed that a bilayer tablet configuration combining roller-compacted granules of naproxen sodium in one layer with acetaminophen as an additional active ingredient in a separate secondary layer demonstrated a significantly shorter disintegration time than comparative monolayer tablets containing naproxen sodium alone or single layer (monolayer) tablets containing naproxen sodium in combination with acetaminophen.

### Primary (or Naproxen Sodium) Layer of Bilayer Tablet

In some embodiments, the bilayer naproxen sodium tablets comprise a primary layer (or naproxen sodium layer), wherein the primary (or naproxen sodium) layer comprises granules comprising naproxen sodium. The intragranular composition of the roller-compacted granules comprising naproxen sodium, with respect to the intragranular excipients and quantities as described herein, may be employed for the granules used in the bilayer naproxen sodium tablets.

In some embodiments, the roller-compacted granules comprise naproxen sodium, mannitol, colloidal silicon dioxide, stearic acid, and sodium starch glycolate. In other embodiments, the roller-compacted granules comprise naproxen sodium, mannitol, colloidal silicon dioxide, sodium stearyl fumarate, and sodium starch glycolate. In some embodiments, the roller-compacted granules comprise naproxen sodium, mannitol, colloidal silicon dioxide, magnesium stearate, and sodium starch glycolate. In yet other embodiments, the roller-compacted granules comprise naproxen sodium, mannitol, colloidal silicon dioxide, stearic acid, sodium stearyl fumarate, and sodium starch glycolate. In some embodiments, the roller-compacted granules comprise naproxen sodium, mannitol, colloidal silicon dioxide, stearic acid, magnesium stearate, and sodium starch glycolate.

In still further embodiments, the bilayer naproxen sodium tablet comprises a combination of mannitol, colloidal silicon dioxide, stearic acid, and sodium starch glycolate as intragranular excipients. In some embodiments, the bilayer naproxen sodium tablet comprises a combination of mannitol, colloidal silicon dioxide, sodium stearyl fumarate, and sodium starch glycolate as intragranular excipients. In other embodiments, the bilayer naproxen sodium tablet comprises a combination of mannitol, colloidal silicon dioxide, stearic acid, sodium stearyl fumarate, and sodium starch glycolate as intragranular excipients. In other embodiments, the bilayer naproxen sodium tablet comprises a combination of mannitol, colloidal silicon dioxide, stearic acid, magnesium stearate, and sodium starch glycolate as intragranular excipients.

In some embodiments, the bilayer naproxen sodium tablet comprises at least 50 mg or at least 100 mg naproxen sodium. In other embodiments, the bilayer naproxen sodium tablet comprises less than or equal to 300 mg, less than or equal to 250 mg, less than or equal to 200 mg, or less than or equal to 150 mg naproxen sodium. In some embodiments, bilayer naproxen sodium tablet comprises between 50 mg and 300 mg, between 50 mg and 250 mg, between 50 mg and 200 mg, between 50 mg and 150 mg, between 100 mg and 300 mg, between 100 mg and 250 mg, between 100 mg and 200 mg, between 150 mg and 300 mg, between 150 mg and 250 mg, between 150 mg and 200 mg, between 200 mg and 300 mg, between 200 mg and 250 mg, or between 250 mg and 300 mg. In certain embodiments, the bilayer naproxen sodium tablet comprises 100 mg, 110 mg, 150 mg, 200 mg, 220 mg, 250 mg, or 300 mg naproxen sodium. In certain other embodiments, the bilayer naproxen sodium tablet comprises 110 mg or 150 mg naproxen sodium.

In some embodiments, the primary (or naproxen sodium) layer comprises at least 10% w/w, at least 12% w/w, at least 15% w/w, at least 17% w/w naproxen sodium of the total weight of the bilayer naproxen sodium tablet. In other embodiments, the primary (or naproxen sodium) layer comprises less than or equal to 30% w/w, less than or equal to 27% w/w, less than or equal to 25% w/w, or less than or equal to 22% w/w naproxen sodium of the total weight of the bilayer naproxen sodium tablet. In some embodiments, the primary (or naproxen sodium) layer comprises 10-30% w/w, 10-27% w/w, 10-25% w/w, 10-22% w/w, 10-20% w/w, 10-17% w/w, 10-15% w/w, 10-12% w/w, 12-30% w/w, 12-27% w/w, 12-25% w/w, 12-22% w/w, 12-20% w/w, 12-17% w/w, 12-15% w/w, 15-30% w/w, 15-27% w/w, 15-25% w/w, 15-22% w/w, 15-20% w/w, 15-17% w/w, 17-30% w/w, 17-27% w/w, 17-25% w/w, 17-22% w/w, 17-20% w/w, 20-30% w/w, 20-27% w/w, 20-25% w/w, 20-22% w/w, 22-30% w/w, 22-27% w/w, 22-25% w/w, 25-30% w/w, 25-27% w/w, or 27-30% w/w naproxen sodium of the total weight of the bilayer naproxen sodium tablet.

In some embodiments, the primary (or naproxen sodium) layer of the bilayer tablet comprises extragranular excipients, including but not limited to lubricants, glidants/flow aids, binders and superdisintegrants.

In some embodiments, the primary (or naproxen sodium) layer comprises one or more extragranular lubricants. In some embodiments, the one or more extragranular lubricants comprises stearic acid, stearyl sodium fumarate, magnesium stearate or any combination thereof. In some embodiments, the primary (or naproxen sodium) layer comprises at least 0.1% w/w, at least 0.5% w/w, or at least 1% w/w extragranular lubricant of the total weight of the bilayer naproxen sodium tablet. In other embodiments, the primary (or naproxen sodium) layer comprises less than or equal to 5% w/w or less than or equal to 2% w/w extragranular lubricant of the total weight of the bilayer naproxen sodium tablet. In certain embodiments, the primary (or naproxen sodium) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w extragranular lubricant of the total weight of the bilayer naproxen sodium tablet. In some embodiments, the primary (or naproxen sodium) layer comprises at least 0.1% w/w, at least 0.5% w/w, or at least 1% w/w extragranular magnesium stearate of the total weight of the bilayer naproxen sodium tablet. In other embodiments, the primary (or naproxen sodium) layer comprises less than or equal to 5% w/w or less than or equal to 2% w/w extragranular magnesium stearate of the total weight of the bilayer naproxen sodium tablet. In certain embodiments, the primary (or naproxen sodium) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w extragranular magnesium stearate of the total weight of the bilayer naproxen sodium tablet. In some embodiments, the primary (or naproxen sodium) layer comprises at least 0.1% w/w, at least 0.5% w/w, or at least 1% w/w extragranular stearic acid of the total weight of the bilayer naproxen sodium tablet. In other embodiments, the primary (or naproxen sodium) layer comprises less than or equal to 5% w/w or less than or equal to 2% w/w extragranular stearic acid of the total weight of the bilayer naproxen sodium tablet. In certain embodiments, the primary (or naproxen sodium) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w extragranular stearic acid of the total weight of the bilayer naproxen sodium tablet.

In some embodiments, the primary (or naproxen sodium) layer comprises one or more glidants or flow aids. In some embodiments, the primary (or naproxen sodium) layer comprises extragranular colloidal silicon dioxide. In some embodiments, the primary (or naproxen sodium) layer comprises at least 0.1% w/w, at least 0.5% w/w, or at least 1% w/w extragranular colloidal silicon dioxide of the total weight of the bilayer naproxen sodium tablet. In other embodiments, the primary (or naproxen sodium) layer comprises less than or equal to 5% w/w or less than or equal to 2% w/w extragranular colloidal silicon dioxide of the total weight of the bilayer naproxen sodium tablet. In certain embodiments, the primary (or naproxen sodium) layer comprises 0.1-5% w/w, 0.1-2% w/w, 0.5-5% w/w, 0.5-2% w/w, 1-5% w/w, or 1-2% w/w extragranular colloidal silicon dioxide of the total weight of the bilayer naproxen sodium tablet.

In some embodiments, the primary (or naproxen sodium) layer comprises one or more binders. Binders may be incorporated in the primary (or naproxen sodium) layer to help maintain adhesion between the granules and other extragranular excipients in the same layer. Suitable binders may include but are not limited to starch or starch derivatives (such as partially pregelatinized starch), or any combination thereof. In some embodiments, the primary (or naproxen sodium) layer comprises starch and/or partially pregelatinized starch. In some embodiments, the primary (or naproxen sodium) layer comprises starch. In some embodiments, the primary (or naproxen sodium) layer comprises at least 0.1% w/w, at least 0.5% w/w, or at least 1% w/w extragranular starch of the total weight of the bilayer naproxen sodium tablet. In other embodiments, the primary (or naproxen sodium) layer comprises less than or equal to 5% w/w or less than or equal to 2% w/w extragranular starch of the total weight of the bilayer naproxen sodium tablet. In certain embodiments, the primary (or naproxen sodium) layer comprises 0.1-5% w/w, 0.1-2% w/w, 0.5-5% w/w, 0.5-2% w/w, 1-5% w/w, or 1-2% w/w extragranular starch of the total weight of the bilayer naproxen sodium tablet. In some embodiments, the primary (or naproxen sodium) layer comprises partially pregelatinized starch. In some embodiments, the primary (or naproxen sodium) layer comprises at least 0.1% w/w, at least 0.5% w/w, or at least 1% w/w extragranular partially pregelatinized starch of the total weight of the bilayer naproxen sodium tablet. In other embodiments, the primary (or naproxen sodium) layer comprises less than or equal to 5% w/w or less than or equal to 2% w/w extragranular partially pregelatinized starch of the total weight of the bilayer naproxen sodium tablet. In certain embodiments, the primary (or naproxen sodium) layer comprises 0.1-5% w/w, 0.1-2% w/w, 0.5-5% w/w, 0.5-2% w/w, 1-5% w/w, or 1-2% w/w extragranular partially pregelatinized starch of the total weight of the bilayer naproxen sodium tablet.

In other embodiments, the primary (or naproxen sodium) layer comprises one or more superdisintegrants as extragranular excipients. In some embodiments, the primary (or naproxen sodium) layer in the bilayer naproxen sodium tablets comprises microcrystalline cellulose (MCC), hydroxypropyl methylcellulose (HPMC) or other cellulose derivatives. In some embodiments, the primary (or naproxen sodium) layer comprises croscarmellose sodium. In some embodiments, the primary (or naproxen sodium) layer comprises extragranular superdisintegrant. In some embodiments, the primary (or naproxen sodium) layer comprises at least 0.1% w/w, at least 0.5% w/w, or at least 1% w/w extragranular superdisintegrant of the total weight of the bilayer naproxen sodium tablet. In other embodiments, the primary (or naproxen sodium) layer comprises less than or equal to 5% w/w or less than or equal to 2% w/w extragranular superdisintegrant of the total weight of the bilayer naproxen sodium tablet. In certain embodiments, the primary (or naproxen sodium) layer comprises 0.1-5% w/w, 0.1-2% w/w, 0.5-5% w/w, 0.5-2% w/w, 1-5% w/w, or 1-2% w/w extragranular superdisintegrant of the total weight of the bilayer naproxen sodium tablet. In some embodiments, the primary (or naproxen sodium) layer comprises extragranular croscarmellose sodium. In some embodiments, the primary (or naproxen sodium) layer comprises at least 0.1% w/w, at least 0.5% w/w, or at least 1% w/w extragranular croscarmellose sodium of the total weight of the bilayer naproxen sodium tablet. In other embodiments, the primary (or naproxen sodium) layer comprises less than or equal to 5% w/w or less than or equal to 2% w/w extragranular croscarmellose sodium of the total weight of the bilayer naproxen sodium tablet. In certain embodiments, the primary (or naproxen sodium) layer comprises 0.1-5% w/w, 0.1-2% w/w, 0.5-5% w/w, 0.5-2% w/w, 1-5% w/w, or 1-2% w/w extragranular croscarmellose sodium of the total weight of the bilayer naproxen sodium tablet.

In some embodiments, the primary (or naproxen sodium) layer comprises a colorant.

### Secondary (or Acetaminophen) Layer of Bilayer Tablet

In some embodiments, the bilayer naproxen sodium tablets provided herein comprise a secondary layer, comprising one or more additional active pharmaceutical ingredients. In some embodiments, the secondary layer which may or may not also include naproxen sodium. In some embodiments, the secondary layer comprises naproxen sodium. In other embodiments, the secondary layer does not contain naproxen sodium.

In some embodiments, the secondary layer comprises one or more additional active pharmaceutical ingredients, wherein the one or more additional active pharmaceutical ingredients comprises acetaminophen. In some embodiments wherein the secondary layer comprises acetaminophen, the secondary layer may alternatively be referred to as an acetaminophen layer.

In some embodiments wherein the secondary layer comprises acetaminophen, the bilayer naproxen sodium tablet comprises at least 50 mg, at least 100 mg, at least 200 mg, at least 300 mg acetaminophen. In other embodiments, the bilayer naproxen sodium tablet comprises less than or equal to 500 mg or less than or equal to 400 mg acetaminophen. In some embodiments, the bilayer naproxen sodium tablet comprises between 50 mg and 500 mg, between 50 mg and 400 mg, between 50 mg and 325 mg, between 50 mg and 200 mg, between 50 mg and 100 mg, between 100 mg and 500 mg, between 100 mg and 400 mg, between 100 mg and 325 mg, between 100 mg and 200 mg, between 200 mg and 500 mg, between 200 mg and 400 mg, between 200 mg and 325 mg, between 325 mg and 500 mg, between 325 mg and 400 mg, or between 400 mg and 500 mg acetaminophen. In certain embodiments, the bilayer naproxen sodium tablet comprises 100 mg, 250 mg, 325 mg, or 500 mg acetaminophen. In certain other embodiments, the bilayer naproxen sodium tablet comprises 325 mg acetaminophen.

In some embodiments, the secondary (or acetaminophen) layer comprises at least 45% w/w, at least 47% w/w, at least 50% w/w, at least 52% w/w, or at least 55% w/w acetaminophen of the total weight of the bilayer naproxen sodium tablet. In some embodiments, the secondary (or acetaminophen) layer comprises less than or equal to 70% w/w, less than or equal to 65% w/w, less than or equal to 60% w/w, or less than or equal to 57% w/w acetaminophen of the total weight of the bilayer naproxen sodium tablet. In some embodiments, the secondary (or acetaminophen) layer comprises 45-70% w/w, 45-65% w/w, 45-60% w/w, 45-57% w/w, 45-55% w/w, 45-52% w/w, 45-50% w/w, 45-47% w/w, 47-70% w/w, 47-65% w/w, 47-60% w/w, 47-57% w/w, 47-55% w/w, 47-52% w/w, 47-50% w/w, 50-70% w/w, 50-65% w/w, 50-60% w/w, 50-57% w/w, 50-55% w/w, 50-52% w/w, 52-70% w/w, 52-65% w/w, 52-60% w/w, 52-57% w/w, 52-55% w/w, 55-70% w/w, 55-65% w/w, 55-60% w/w, 55-57% w/w, 57-70% w/w, 57-65% w/w, 57-60% w/w, 60-70% w/w, 60-65% w/w, or 65-70% w/w acetaminophen of the total weight of the bilayer naproxen sodium tablet.

It should be recognized that the other active pharmaceutical ingredients may be suitably employed as the one or more additional active pharmaceutical ingredients in the secondary layer of the bilayer naproxen sodium tablets, either in lieu of or in combination with acetaminophen. It should be further recognized that active pharmaceutical ingredients that demonstrate a similar disintegration mechanism as acetaminophen or comparably quick disintegration time as observed for the acetaminophen layer described herein may also provide a final bilayer naproxen sodium tablet exhibiting short disintegration times.

In some embodiments, the secondary (or acetaminophen) layer comprises one or more binders. Binders may be incorporated in the secondary (or acetaminophen) layer to help maintain adhesion between the active pharmaceutical ingredient(s), such as acetaminophen, and the excipients in the same layer. Suitable binders may include but are not limited to starch or starch derivatives (such as partially pregelatinized starch), or any combination thereof.

In some embodiments, the secondary (or acetaminophen) layer comprises starch. In some embodiments, the secondary (or acetaminophen) layer comprises at least 2% w/w, at least 3% w/w, at least 4% w/w, or at least 5% w/w starch of the total weight of the bilayer naproxen sodium tablet. In other embodiments, the secondary (or acetaminophen) layer comprises less than or equal to 15% w/w, less than or equal to 12% w/w, or less than or equal to 10% w/w starch of the total weight of the bilayer naproxen sodium tablet. In some embodiments, the secondary (or acetaminophen) layer comprises 2-15% w/w, 2-12% w/w, 2-10% w/w, 2-7% w/w, 2-5% w/w, 2-4% w/w, 2-3% w/w, 3-15% w/w, 3-12% w/w, 3-10% w/w, 3-7% w/w, 3-5% w/w, 3-4% w/w, 4-15% w/w, 4-12% w/w, 4-10% w/w, 4-7% w/w, 4-5% w/w, 5-15% w/w, 5-12% w/w, 5-10% w/w, 5-7% w/w, 7-15% w/w, 7-12% w/w, 7-10% w/w, 10-15% w/w, 10-12% w/w, or 12-15% w/w starch of the total weight of the bilayer naproxen sodium tablet.

In some embodiments, the secondary (or acetaminophen) layer comprises partially pregelatinized starch. In some embodiments, the secondary (or acetaminophen) layer comprises at least 2% w/w, at least 3% w/w, at least 4% w/w, or at least 5% w/w partially pregelatinized starch of the total weight of the bilayer naproxen sodium tablet. In other embodiments, the secondary (or acetaminophen) layer comprises less than or equal to 15% w/w, less than or equal to 12% w/w, or less than or equal to 10% w/w partially pregelatinized starch of the total weight of the bilayer naproxen sodium tablet. In some embodiments, the secondary (or acetaminophen) layer comprises 2-15% w/w, 2-12% w/w, 2-10% w/w, 2-7% w/w, 2-5% w/w, 2-4% w/w, 2-3% w/w, 3-15% w/w, 3-12% w/w, 3-10% w/w, 3-7% w/w, 3-5% w/w, 3-4% w/w, 4-15% w/w, 4-12% w/w, 4-10% w/w, 4-7% w/w, 4-5% w/w, 5-15% w/w, 5-12% w/w, 5-10% w/w, 5-7% w/w, 7-15% w/w, 7-12% w/w, 7-10% w/w, 10-15% w/w, 10-12% w/w, or 12-15% w/w partially pregelatinized starch of the total weight of the bilayer naproxen sodium tablet.

In some embodiments, the secondary (or acetaminophen) layer comprises starch and partially pregelatinized starch. In some embodiments, the secondary (or acetaminophen) layer comprises at least 2% w/w, at least 3% w/w, at least 4% w/w, or at least 5% w/w starch and partially pregelatinized starch of the total weight of the bilayer naproxen sodium tablet. In other embodiments, the secondary (or acetaminophen) layer comprises less than or equal to 15% w/w, less than or equal to 12% w/w, or less than or equal to 10% w/w starch and partially pregelatinized starch of the total weight of the bilayer naproxen sodium tablet. In some embodiments, the secondary (or acetaminophen) layer comprises 2-15% w/w, 2-12% w/w, 2-10% w/w, 2-7% w/w, 2-5% w/w, 2-4% w/w, 2-3% w/w, 3-15% w/w, 3-12% w/w, 3-10% w/w, 3-7% w/w, 3-5% w/w, 3-4% w/w, 4-15% w/w, 4-12% w/w, 4-10% w/w, 4-7% w/w, 4-5% w/w, 5-15% w/w, 5-12% w/w, 5-10% w/w, 5-7% w/w, 7-15% w/w, 7-12% w/w, 7-10% w/w, 10-15% w/w, 10-12% w/w, or 12-15% w/w starch and partially pregelatinized starch of the total weight of the bilayer naproxen sodium tablet.

As described herein, acetaminophen demonstrates disintegration behavior that is directly correlated to the presence of disintegrants and/or superdisintegrants. In the bilayer naproxen sodium tablets described herein, the incorporation of disintegration aids in the secondary (acetaminophen) layer contributes significantly to the rapid disintegration time observed for the bilayer tablet as a whole. In other embodiments, the secondary (or acetaminophen) layer comprises one or more superdisintegrants. In some embodiments, the secondary (or acetaminophen) layer comprises at least 1% w/w, at least 2% w/w, or at least 3% w/w superdisintegrant of the total weight of the bilayer naproxen sodium tablet. In other embodiments, the secondary (or acetaminophen) layer comprises less than or equal to 6% w/w, less than or equal to 5% w/w, or less than or equal to 4% w/w superdisintegrant of the total weight of the bilayer naproxen sodium tablet. In some embodiments, the secondary (or acetaminophen) layer comprises 1-6% w/w, 1-5% w/w, 1-4% w/w, 1-3% w/w, 1-2% w/w, 2-6% w/w, 2-5% w/w, 2-4% w/w, 2-3% w/w, 3-6% w/w, 3-5% w/w, 3-4% w/w, 4-6% w/w, 4-5% w/w, or 5-6% w/w superdisintegrant of the total weight of the bilayer naproxen sodium tablet. In some embodiments, the secondary (or acetaminophen) layer comprises at least 1% w/w, at least 2% w/w, or at least 3% w/w croscarmellose sodium of the total weight of the bilayer naproxen sodium tablet. In other embodiments, the secondary (or acetaminophen) layer comprises less than or equal to 6% w/w, less than or equal to 5% w/w, or less than or equal to 4% w/w croscarmellose sodium of the total weight of the bilayer naproxen sodium tablet. In some embodiments, the secondary (or acetaminophen) layer comprises 1-6% w/w, 1-5% w/w, 1-4% w/w, 1-3% w/w, 1-2% w/w, 2-6% w/w, 2-5% w/w, 2-4% w/w, 2-3% w/w, 3-6% w/w, 3-5% w/w, 3-4% w/w, 4-6% w/w, 4-5% w/w, or 5-6% w/w croscarmellose sodium of the total weight of the bilayer naproxen sodium tablet.

In some embodiments, the secondary (or acetaminophen) layer comprises one or more glidants or flow aids. In some embodiments, the secondary (or acetaminophen) layer comprises colloidal silicon dioxide. In some embodiments, the secondary (or acetaminophen) layer comprises at least 0.1% w/w, at least 0.5% w/w, or at least 1% w/w colloidal silicon dioxide of the total weight of the bilayer naproxen sodium tablet. In other embodiments, the secondary (or acetaminophen) layer comprises less than or equal to 5% w/w or less than or equal to 2% w/w colloidal silicon dioxide of the total weight of the bilayer naproxen sodium tablet. In certain embodiments, the secondary (or acetaminophen) layer comprises 0.1-5% w/w, 0.1-2% w/w, 0.5-5% w/w, 0.5-2% w/w, 1-5% w/w, or 1-2% w/w colloidal silicon dioxide of the total weight of the bilayer naproxen sodium tablet.

In some embodiments, the secondary (or acetaminophen) layer comprises at least 0.1% w/w, at least 0.5% w/w, or at least 1% w/w lubricant of the total weight of the bilayer naproxen sodium tablet. In other embodiments, the secondary (or acetaminophen) layer comprises less than or equal to 5% w/w or less than or equal to 2% w/w lubricant of the total weight of the bilayer naproxen sodium tablet. In certain embodiments, the secondary (or acetaminophen) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w lubricant of the total weight of the bilayer naproxen sodium tablet. In some embodiments, the secondary (or acetaminophen) layer comprises at least 0.1% w/w, at least 0.5% w/w, or at least 1% w/w magnesium stearate of the total weight of the bilayer naproxen sodium tablet. In other embodiments, the secondary (or acetaminophen) layer comprises less than or equal to 5% w/w or less than or equal to 2% w/w magnesium stearate of the total weight of the bilayer naproxen sodium tablet. In certain embodiments, the secondary (or acetaminophen) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w magnesium stearate of the total weight of the bilayer naproxen sodium tablet. In some embodiments, the secondary (or acetaminophen) layer comprises at least 0.1% w/w, at least 0.5% w/w, or at least 1% w/w stearic acid of the total weight of the bilayer naproxen sodium tablet. In other embodiments, the secondary (or acetaminophen) layer comprises less than or equal to 5% w/w or less than or equal to 2% w/w stearic acid of the total weight of the bilayer naproxen sodium tablet. In certain embodiments, the secondary (or acetaminophen) layer comprises comprise 0.1-10% w/w, 0.1-5% w/w, 0.1-2% w/w, 0.5-10% w/w, 0.5-5% w/w, 0.5-2% w/w, 1-10% w/w, 1-5% w/w, or 1-2% w/w stearic acid of the total weight of the bilayer naproxen sodium tablet.

In some embodiments, the secondary (or acetaminophen) layer comprises a colorant.

In some embodiments, the one or more extragranular lubricants in the primary (or naproxen sodium) layer and the one or more lubricants in the secondary (or acetaminophen) layer are the same. In some embodiments, the one or more extragranular superdisintegrants in the primary (or naproxen sodium) layer and the one or more superdisintegrants in the secondary (or acetaminophen) layer are the same.

In some embodiments, the bilayer naproxen sodium tablet further comprises a film coating. In some variations, the film coating comprises poly(vinylalcohol). In certain embodiments, the film coating is an immediate release coating. In other embodiments, the film coating further comprises a colorant, a flavorant, or a combination thereof.

### Tablet Dissolution, Disintegration and Other Properties of the Bilayer Tablet

In some embodiments, the bilayer naproxen sodium tablets provided herein may be characterized by their disintegration and/or dissolution properties. As described herein, the bilayer naproxen sodium tablets demonstrate unexpectedly short disintegration times.

The disintegration time of bilayer naproxen sodium tablets of the present disclosure may be determined by the USP Disintegration Test in water at 37° C.±0.5° C. using a basket-rack assembly with disks. For example, in some embodiments, the bilayer naproxen sodium tablet has a disintegration time of less than about 8 minutes, less than about 7 minutes, less than about 6 minutes, less than about 5 minutes, or less than about 4 minutes as determined by USP Disintegration Test in water at 37° C.±0.5° C. using a basket-rack assembly with disks. In other embodiments, the bilayer naproxen sodium tablet has a disintegration time of at least about 1 minute, at least about 2 minutes or at least about 3 minutes as determined by the USP Disintegration Test in water at 37° C.±0.5° C. using a basket-rack assembly with disks. In certain embodiments, the bilayer naproxen sodium tablet has a disintegration time of between 1 minute and 8 minutes, between 1 minutes and 7 minutes, between 1 minute and 6 minutes, between 1 minute and 5 minutes, between 1 minute and 4 minutes, between 2 minutes and 8 minutes, between 2 minutes and 7 minutes, between 2 minutes and 6 minutes, between 2 minutes and 5 minutes, between 2 minutes and 4 minutes, between 3 minutes and 8 minutes, between 3 minutes and 7 minutes, between 3 minutes and 6 minutes, between 3 minutes and 5 minutes, between 3 minutes and 4 minutes, as determined by the USP disintegration test in water at 37° C.±0.5° C. using a basket-rack assembly with disks. In other embodiments, the bilayer naproxen sodium tablet is not an orally disintegrating tablet.

The bilayer naproxen sodium tablets may also be characterized by their dissolution profiles as determined by the USP Apparatus-2 Dissolution Test described herein. As described above for monolayer naproxen sodium tablets, the bilayer naproxen sodium tablets may be further characterized by a number of properties associated with their physical durability and structural integrity.

In some embodiments, the naproxen bilayer sodium tablet has a hardness of at least 2 kilopond (kp), at least 3 kp, at least 4 kp, at least 5 kp, or at least 6 kp as determined by a tablet tester in accordance with the USP Tablet Breaking Force Test. In other embodiments, the bilayer naproxen sodium tablet has a hardness of less than or equal to 18 kp, less than or equal to 17 kp, less than or equal to 16 kp, less than or equal to 15 kp, less than or equal to 14 kp, less than or equal to 13 kp, or less than or equal to 12 kp as determined by a tablet tester in accordance with the USP Tablet Breaking Force Test. In certain embodiments, the bilayer naproxen sodium tablet has a hardness between 2 and 18 kp, between 2 and 16 kp, between 2 and 14 kp, between 2 and 12 kp, between 4 and 18 kp, between 4 and 16 kp, between 4 and 14 kp, between 4 and 12 kp, between 6 and 18 kp, between 6 and 16 kp, between 6 and 14 kp, or between 6 and 12 kp as determined by a tablet tester in accordance with the USP Tablet Breaking Force Test.

In some embodiments, the bilayer naproxen sodium tablet has a friability of at least 0.1%, at least 0.2%, at least 0.3%, at least 0.4%, or at least 0.5% as determined by the USP Friability Test after 200 revolutions. In other embodiments, the bilayer naproxen sodium tablet has a friability of less than or equal to 1%, less than or equal to 0.9%, less than or equal to 0.8%, less than or equal to 0.7%, less than or equal to 0.6%, or less than or equal to 0.5% as determined by the USP Friability Test after 200 revolutions. In certain embodiments, the bilayer naproxen sodium tablet has a friability of between 0.1% and 1%, between 0.1% and 0.9%, between 0.1% and 0.7%, between 0.1% and 0.5%, between 0.3% and 1%, between 0.3% and 0.9%, between 0.3% and 0.7%, between 0.3% and 0.5%, between 0.5% and 1%, between 0.5% and 0.9%, or between 0.5% and 0.7% as determined by the USP Friability Test after 200 revolutions.

### Methods of Preparing Oral Tablets

As described herein the oral tablets, and, more specifically, the naproxen sodium tablets, of the present disclosure are prepared by a dry granulation process rather than a wet granulation process. In one aspect, the present disclosure provides methods of preparing the oral tablets comprising roller-compacted granules as described herein.

Granulation is a method of combining and converting individual powder components into pre-formed aggregate or agglomerated particles (granules) containing two or more powder components and having well-defined size distributions, which help to ensure the consistency of tableting and/or other mechanical treatments later in the manufacturing process. Dry granulation by roller compaction is a process of passing a mixture of an active pharmaceutical ingredient and dry excipients through a pair of rollers to compress the powder into sheets or ribbons, which are subsequently milled or ground into granules. Following granulation, the granules are combined with additional excipients to be compressed into a tablet form. Dry granulation does not require the use of a “wet” granulation fluid to combine excipients at the granulation stage, and consequently does not require downstream drying to remove residual moisture of granulation fluid.

Dry granulation can be a complex procedure due to the diversity of excipients available for blending with the active pharmaceutical ingredient, as well as the different adjustment parameters in the roller compaction process that can influence the properties of the final product. For example, the choice of excipients, roll speed, roll gap/nip angle, and feed speed are a few of the variables that may affect the density of the compacted ribbons. The production of consistently uniform ribbons, with specific ribbon densities, may influence the ability to obtain reproducible granules (size distribution, porosity). The reproducibility of the granule production process may further affect the homogeneity, compressibility, and compactability of the material used in downstream mixing and tableting processes, which in turn can influence dissolution profiles, disintegration times, and hardness of the tablets. Thus, the control of process parameters and other variables in processes involving dry granulation may be important to ensure tablet quality and reproducibility.

The formulation of naproxen sodium with pharmaceutically acceptable excipients as described herein was found to be uniquely compatible with roller compaction and tableting, in terms of flowability, compressibility and minimal loss of material (due to sticking/picking). Moreoever, the composition of the intragranular excipients was found be readily under a range of process parameters while still resulting in roller-compacted granules with consistent porosity and particle size distribution and, ultimately, in a highly reproducible naproxen sodium tablet having enhanced dissolution and disintegration.

In one aspect, provided herein is a process for preparing a naproxen sodium tablet, comprising: combining naproxen sodium, mannitol, colloidal silicon dioxide, one or more lubricants, and one or more superdisintegrants to provide a blend mixture; compacting the blend mixture by roller compaction to provide ribbons; milling the ribbons to provide granules; combining the granules with mannitol, one or more lubricants, one or more superdisintegrants, and optionally colloidal silicon dioxide, to provide a tableting mixture; and compressing the tableting mixture to provide the naproxen sodium tablet.

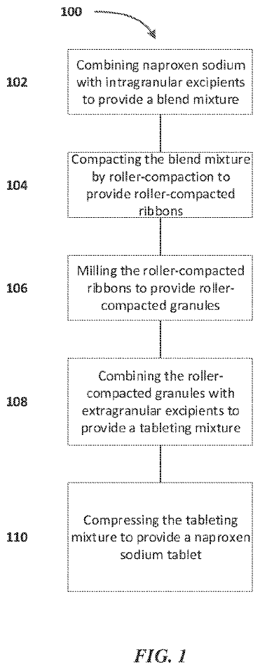

With reference to FIG. 1, process 100 is an exemplary process for preparing a naproxen sodium tablet. In step 102, naproxen sodium is combined with intragranular excipients (e.g., mannitol, colloidal silicon dioxide, stearic acid, and sodium starch glycolate) to form a blend mixture. The naproxen sodium and intragranular excipients are provided in dry powder forms. The resulting blend mixture is processed in steps 104 and 106 to provide granulated naproxen sodium. The blend mixture comprising naproxen sodium and intragranular excipients are compacted by roller compaction to provide ribbons in step 104. In step 106, the ribbons are milled to provide roller-compacted granules. The roller-compacted granules are then combined with extragranular excipients (e.g., mannitol, sodium starch glycolate, and magnesium stearate, and optionally colloidal silicon dioxide) to provide a tableting mixture in step 108. The tablet mixture is then compressed in step 110 to give a naproxen sodium tablet.

In another aspect, provided herein is a method of preparing a naproxen sodium tablet, comprising: combining naproxen sodium, mannitol, colloidal silicon dioxide, stearic acid and sodium starch glycolate to provide a blend mixture; compacting the blend mixture by roller compaction to provide ribbons; milling the ribbons to provide granules; combining the granules with mannitol, sodium starch glycolate, magnesium stearate, and optionally colloidal silicon dioxide, to provide a tableting mixture; and compressing the tableting mixture to provide the naproxen sodium tablet.

Notably, the components described herein for the naproxen sodium tablets are compatible with dry granulation by roller-compaction and subsequent compression to provide a tablet having enhanced dissolution with minimal material loss throughout the manufacturing process. The particular selection of intragranular and extragranular excipients for the naproxen sodium tablets described herein also surprisingly results in tablets having an enhanced dissolution profile that remains consistent even when processing parameters are adjusted.

For example, the compaction of the blend mixture by roller compaction will typically result in roller-compacted ribbons having varied properties depending upon the conditions under which the blend mixture was compacted. Process parameters for roller compaction may include, but are not limited to, the feed rate of the blend mixture into the roller compactor, the type of rollers employed (smooth and/or serrated), the roller speed, the roller gap, and the roller pressure. The properties of the resulting ribbons that are affected by these variables include but are not limited to the porosity, the solid fraction, the hardness, and/or the thickness.

As described in the foregoing methods, following preparation of the blend mixture, the blend mixture is subjected to roller compaction. With reference to FIG. 1, step 104, in some embodiments, the blend mixture is compacted by roller compaction at variable process settings, including for example, the applied pressure of the rollers (e.g., between 18 and 30 bar), the roller speed (e.g., between 4 and 9 rpm), and the roller gap (e.g., between 1.0 and 4.0 mm).

The applied pressure, roller speed, and roller gap may all influence the resulting hardness, thickness and porosity of the resulting roller-compacted material. In some embodiments of the foregoing methods, the blend mixture is compacted by roller compaction at an applied force of at least 10 bar, at least 15 bar, or at least 18 bar. In other embodiments, the blend mixture is compacted by roller compaction at an applied force of less than or equal to 40 bar, less than or equal to 35 bar, or less than or equal to 30 bar. In certain embodiments, the blend mixture is compacted by roller compaction at an applied force of between 10 and 40 bar, between 10 and 35 bar, between 10 and 30 bar, between 15 and 40 bar, between 15 and 35 bar, between 15 and 30 bar, between 18 and 40 bar, between 18 and 35 bar, or between 18 and 30 bar.

In some embodiments, the blend mixture is compacted by roller compaction at a roller speed of at least 1 rpm, at least 2 rpm, at least 3 rpm, or at least 4 rpm. In other embodiments, the blend mixture is compacted by roller compaction at a roller speed of less than or equal 12 rpm, less than or equal 11 rpm, less than or equal 10 rpm, or less than or equal 9 rpm. In certain embodiments, the blend mixture is compacted by roller compaction at a roller speed of between 1 rpm and 12 rpm, between 1 rpm and 11 rpm, between 1 rpm and 10 rpm, between 1 rpm and 9 rpm, between 2 rpm and 12 rpm, between 2 rpm and 11 rpm, between 2 rpm and 10 rpm, between 2 rpm and 9 rpm, between 3 rpm and 12 rpm, between 3 rpm and 11 rpm, between 3 rpm and 10 rpm, between 3 rpm and 9 rpm, between 4 rpm and 12 rpm, between 4 rpm and 11 rpm, between 4 rpm and 10 rpm, or between 4 rpm and 9 rpm.

In still other embodiments, the blend mixture is compacted by roller compaction at a roller gap of at least 0.5 mm, or at least 1 mm, or at least 1.5 mm. In yet other embodiments, the blend mixture is compacted by roller compaction at a roller gap of less than or equal to 6 mm, less than or equal to 5 mm, or less than or equal to 4 mm. In certain embodiments, the blend mixture is compacted by roller compaction at a roller gap of between 0.5 and 6 mm, between 0.5 and 5 mm, between 0.5 and 4 mm, between 1 and 6 mm, between 1 and 5 mm, between 1 and 4 mm, between 1.5 and 6 mm, between 1.5 and 5 mm, or between 1.5 and 4 mm.

In addition to the foregoing roller parameters, it should be recognized that the compaction of the blend mixture by roller compaction may be further carried with smooth and/or serrated rollers. In some embodiments, the rollers employed in the roller compaction of the blend mixture are smooth rollers, serrated rollers, or a combination thereof.

It should be understood that the roller compaction of the powder blend mixture in step 104 may result in an output of compacted, or densified, material, the shape of which may vary depending upon the surface profile of the rollers. In some embodiments, the compacted, or densified powder blend may be formed into rectangular sheets, which may be referred to as ribbons or roller-compacted ribbons. The resulting ribbons may be characterized, for example, by solid fraction, porosity, hardness, and/or thickness.

In some embodiments, the roller-compacted ribbons may be characterized by their solid fraction (or relative density). The solid fraction is a measure of percentage of the bulk volume of a material that is occupied by solid material rather than void space or pores. The solid fraction can be calculated as the ratio of the envelope density (Pₑ) of the material to the true density (Pₒ) of the material (SF=Pₑ/Pₒ). The envelope density is measured as the displacement of a solid medium that can conform to the surface of the material under investigation but does not insert into void space or pores; the true density is measured by gas displacement and reflects the solid volume of a material accounting for void space and pores. In some embodiments, the roller-compacted ribbons have a solid fraction of at least 0.4, at least 0.45, at least 0.5, at least 0.55 or at least 0.6. In other embodiments, the roller compacted ribbons have a solid fraction of less than or equal to 0.9, less than or equal to 0.85, less than or equal to 0.8, less than or equal to 0.75, or less than or equal to 0.7. In certain embodiments, the roller-compacted ribbons have a solid fraction between 0.4 and 0.9, between 0.4 and 0.8, between 0.4 and 0.7, between 0.5 and 0.9, between 0.5 and 0.8, between 0.5 and 0.7, between 0.6 and 0.9, between 0.6 and 0.8, or between 0.6 and 0.7.

The roller-compacted ribbons of the foregoing methods may also be characterized by their porosity. Porosity is a measure of void space (that is not occupied by solids) within a material. The porosity may be calculated from the solid fraction (SF) by the following equation: porosity=[1-SF]×100%. In some embodiments, the roller-compacted ribbons have a porosity of at least 10%, at least 15%, at least 20%, at least 25% or at least 30%. In still other embodiments, the roller-compacted ribbons have a porosity of less than or equal to 60%, less than or equal to 55%, less than or equal to 50%, less than or equal to 45% or less than or equal to 40%. In certain embodiments, the roller-compacted ribbons have a porosity between 10% and 60%, between 10% and 50%, between 10% and 40%, between 20% and 60%, between 20% and 50%, between 20% and 40%, between 30% and 60%, between 30% and 50%, or between 30% and 40%.

The roller-compacted ribbons may be further characterized by their hardness and/or thickness, which may be determined by methods known in the art. Thickness of the roller-compacted ribbons may be determined by measurement with a vernier caliper. Hardness of the ribbons may be measured using snap, bend, and/or break test methods with a hardness-measuring instrument configured accordingly. For example, a suitable method for measuring hardness may involve an instrument with a three-point bend set-up or configuration. In the three-point set-up, a segment of the ribbon to be evaluated is placed on top of two supporting “fulcrums” on either end of the ribbon segment; a third, central “fulcrum” is positioned above the ribbon segment and manipulated to apply a downward force to induce bending/breaking.

It should be recognized that the roller-compacted ribbons of the foregoing methods may possess one or more of the aforementioned characteristics in combination.

Following the production of roller-compacted ribbons, the dry granulation process is completed with a milling step to convert the ribbons into a mass of roller-compacted granules. In some embodiments, the ribbons are milled at a mill speed of at least 40 rpm, 60 rpm, 80 rpm or 100 rpm. In other embodiments, the ribbons are milled at a mill speed of less than or equal to 160 rpm, less than or equal to 140 rpm, less than or equal to 120 rpm, or less than or equal to 100 rpm. In certain embodiments, the ribbons are milled at a mill speed of between 40 and 160 rpm, between 40 and 140 rpm, between 40 and 120 rpm, between 40 and 100 rpm, between 60 and 160 rpm, between 60 and 140 rpm, between 60 and 120 rpm, between 60 and 100 rpm, between 80 and 160 rpm, between 80 and 140 rpm, between 80 and 120 rpm, between 80 and 100 rpm, between 100 and 160 rpm, between 100 and 140 rpm, or between 100 and 120 rpm.

In some embodiments of the foregoing method, the method further comprises sieving the roller-compacted granules to provide roller-compacted granules having a particular particle size distribution. However, in the methods of the present disclosure, it was observed that the roller-compacted granules produced by the milling step were observed to have consistent particle size distribution, bulk density and tapped density across a range of roller compaction parameters and/or milling speeds.

As suggested by the name, dry granulation methods are carried out without the use of “wet” granulation fluids, such as water or ethanol, to help with the mixing and compaction of the active ingredient and excipients to form the granules. Thus, the roller-compacted granules do not require a drying step prior to tableting to remove excess moisture.

As described above, a granule may be characterized as a solid aggregate or agglomerated particle formed from two or more fine powder materials into a single mass. Although a granule is an aggregate mass, it should be understood that a granule may be characterized as a particle, and a plurality of granules may be further characterized by a particle (or granule) size distribution or other particle size attributes.

Accordingly, the granules produced in the methods and comprising naproxen sodium and the intragranular excipients described herein may be characterized by their particle size attributes to distinguish them from the naproxen sodium and/or intragranular excipients in their unmixed, original fine powder forms. In still further embodiments of the foregoing methods, the granules may be characterized by their particle size attributes (e.g., average particle size, particle size distribution, particle size range, etc.).

Particle size distributions of the roller-compacted granules may be determined by methods known in the art, including, for example, sieve analysis using a mechanical sifter (e.g., successive applications of a series of sieves or mesh material) or a laser diffraction particle size analyzer to quantify the amount of material for a given particle size range (i.e., a mass percentage distribution). In some embodiments, roller-compacted granules have a particle size distribution with at least 20%, at least 30%, at least 40%, or at least 50% of the particles having a particle size greater than or equal to 250 μm. In other embodiments, the roller compacted granules have a particle size distribution with less than or equal 90%, less than or equal to 80%, less than or equal to 70% of the particles having a particle size greater than or equal to 250 μm. In certain embodiments, the roller-compacted granules have a particle size distribution with between 20% and 90%, between 20% and 80%, between 20% and 70%, between 30% and 90%, between 30% and 80%, between 30% and 70%, between 40% and 90%, between 40% and 80%, between 40% and 70%, between 50% and 90%, between 50% and 80%, or between 50% and 70% having a particle size greater than or equal to 250 μm.

In addition to particle size distribution, the bulk density and tapped density for the roller-compacted granules can be indicative of a material's ability to settle, to flow, and/or to be compressed. Bulk and tapped densities may be determined by methods known in the art. For example, a given mass of material may be placed into a cylindrical volumetric measuring vessel, allowed to settle, the volume occupied measured and the resulting density calculated as the bulk density. The same mass of material may then be tapped with slight mechanical force, e.g., allowed to fall from a specified controlled height, and the resulting density calculated as the tapped density. In some embodiments, the roller-compacted granules have a bulk density of at least 0.3 g/cc, at least 0.4 g/cc, or at least 0.5 g/cc. In other embodiments the roller-compacted granules have a bulk density of less than or equal to 0.9 g/cc, less than or equal to 0.8 g/cc, or less than or equal to 0.7 g/cc. In certain embodiments, the roller-compacted granules have a bulk density of between 0.3 and 0.9 g/cc, between 0.3 and 0.8 g/cc, between 0.3 and 0.7 g/cc, between 0.4 and 0.9 g/cc, between 0.4 and 0.8 g/cc, between 0.4 and 0.7 g/cc, between 0.5 and 0.9 g/cc, between 0.5 and 0.8 g/cc, or between 0.5 and 0.7 g/cc.

In still other embodiments, the roller-compacted granules have a tapped density of at least 0.5 g/cc, at least 0.6 g/cc, at least 0.7 g/cc. In some embodiments, the roller-compacted granules have a tapped density of less than or equal to 0.95 g/cc, less than or equal to 0.9 g/cc, or less than or equal to 0.8 g/cc. In certain embodiments, the roller-compacted granules have a tapped density of between 0.5 and 0.95 g/cc, between 0.5 and 0.9 g/cc, between 0.5 and 0.8 g/cc, between 0.6 and 0.95 g/cc, between 0.6 and 0.9 g/cc, between 0.6 and 0.8 g/cc, between 0.7 and 0.95 g/cc, between 0.7 and 0.9 g/cc, or between 0.7 and 0.8 g/cc.

The roller-compacted granules may be further characterized by their compressibility, which may be calculated as Compressibility=Tapped Density−Bulk Density/Tapped Density)×100. In some embodiments of the foregoing methods, the roller-compacted granules have a compressibility of at least 5%, at least 10%, or at least 15%. In other embodiments, the roller-compacted granules have a compressibility less than or equal to 30%, less than or equal to 25%, or less than or equal to 20%. In certain embodiments, the roller-compacted granules have a compressibility between 5% and 30%, between 5% and 25%, between 5% and 20%, between 10% and 30%, between 10% and 25%, between 10% and 20%, between 15% and 30%, or between 15% and 25%.

The tableting mixture of the foregoing methods may be characterized by a number of measurable properties including but not limited to particle size distribution, bulk density, tapped density, compressibility, and/or flowability. These properties may influence the dissolution and disintegration properties of the resulting tablets. Particle size distribution, bulk density, and tapped density for the final tableting mixture may be determined as described above for the roller-compacted granules.

Particle size distributions of the tableting mixture may be determined by similar methods as described above for assessing the particle size distribution of the roller-compacted granules--for example, successive applications of a series of sieves or mesh material. In some embodiments, the tableting mixture has a particle size distribution with at least 20%, at least 30%, or at least 40%, of the particles having a particle size greater than or equal to 250 μm. In other embodiments, the tableting mixture has a particle size distribution with less than or equal 90%, less than or equal to 80%, less than or equal to 70% of the particles having a particle size greater than or equal to 250 μm. In certain embodiments, the tableting mixture has a particle size distribution with between 20% and 90%, between 20% and 80%, between 20% and 70%, between 30% and 90%, between 30% and 80%, between 30% and 70%, between 40% and 90%, between 40% and 80%, or between 40% and 70%, having a particle size greater than or equal to 250 μm.

As described above, the bulk and tapped densities of the tableting mixture may be similarly measured by methods known in the art. In some embodiments, the tableting mixture has a bulk density of at least 0.3 g/cc, at least 0.4 g/cc, or at least 0.5 g/cc. In other embodiments the tableting mixture has a bulk density of less than or equal to 0.9 g/cc, less than or equal to 0.8 g/cc, or less than or equal to 0.7 g/cc. In certain embodiments, the tableting mixture has a bulk density of between 0.3 and 0.9 g/cc, between 0.3 and 0.8 g/cc, between 0.3 and 0.7 g/cc, between 0.4 and 0.9 g/cc, between 0.4 and 0.8 g/cc, between 0.4 and 0.7 g/cc, between 0.5 and 0.9 g/cc, between 0.5 and 0.8 g/cc, or between 0.5 and 0.7 g/cc.

In still other embodiments, the tableting mixture has a tapped density of at least 0.5 g/cc, at least 0.6 g/cc, at least 0.7 g/cc. In some embodiments, the tableting mixture has a tapped density of less than or equal to 0.95 g/cc, less than or equal to 0.9 g/cc, or less than or equal to 0.8 g/cc. In certain embodiments, the tableting mixture has a tapped density of between 0.5 and 0.95 g/cc, between 0.5 and 0.9 g/cc, between 0.5 and 0.8 g/cc, between 0.6 and 0.95 g/cc, between 0.6 and 0.9 g/cc, between 0.6 and 0.8 g/cc, between 0.7 and 0.95 g/cc, between 0.7 and 0.9 g/cc, or between 0.7 and 0.8 g/cc.

In some embodiments of the foregoing methods, the tableting mixture has a compressibility of at least 5%, at least 10%, or at least 15%. In other embodiments the tableting mixture has a compressibility less than or equal to 30%, less than or equal to 25%, or less than or equal to 20%. In certain embodiments, the tableting mixture has a compressibility between 5% and 30%, between 5% and 25%, between 5% and 20%, between 10% and 30%, between 10% and 25%, between 10% and 20%, between 15% and 30%, or between 15% and 25%.

In the foregoing methods, the tableting mixture is compressed, which may be carried out by any suitable tablet press. In some embodiments, the compression force applied to the tableting mixture may be varied. For example, in some embodiments the tableting mixture is compressed with a compression force of at least 6 kN, or at least 10 kN, or at least 15 kN. In other embodiments, the tableting mixture is compressed with a compression force of less than or equal to 30 kN, less than or equal to 25 kN, or less than or equal to 20 kN. In certain embodiments, the tableting mixture is compressed with a compression force between 6 and 30 kN, between 6 and 25 kN, between 6 and 20 kN, between 10 and 30 kN, between 10 and 25 kN, between 10 and 20 kN, between 15 and 30 kN, between 15 and 25 kN, or between 15 and 20 kN to provide the naproxen sodium tablet.

In some embodiments of the foregoing method, the method further comprises coating the naproxen sodium tablet to provide a coated naproxen sodium tablet.

In some embodiments, one or more steps of the foregoing method may be carried out as a continuous process or a batch process.

In still yet another aspect, provided herein are methods for preparing bilayer naproxen sodium tablets containing granules comprising naproxen sodium and one or more additional active pharmaceutical ingredients as described herein. In some embodiments, the methods for preparing the bilayer naproxen sodium tablets comprises similar intragranular excipients and similar steps as described above for the preparation of roller-compacted granules comprising naproxen sodium to form the primary (or naproxen sodium) layer of the bilayer tablet. The methods for preparing the bilayer naproxen sodium tablets described herein further combines the preparation of roller-compacted granules and the tableting mixture for the naproxen sodium layer with a parallel preparation of one or more additional active pharmaceutical agents, such as acetaminophen, and excipients, including superdisintegrants, to serve as the tableting mixture to form the secondary layer of the bilayer tablet.

In one aspect, provided herein is a method of preparing a bilayer naproxen sodium tablet, comprising: combining naproxen sodium, mannitol, colloidal silicon dioxide, one or more lubricants and one or more superdisintegrants to provide a blend mixture; compacting the blend mixture by roller compaction to provide ribbons; milling the ribbons to provide granules; combining the granules with mannitol, one or more binders, one or more lubricants, one or more superdisintegrants, and optionally colloidal silicon dioxide, to provide a primary tableting mixture; combining one or more additional active pharmaceutical ingredients, colloidal silicon dioxide, one or more lubricants, and one or more superdisintegrants, and optionally one or more binders or compression aids, to provide a secondary tableting mixture; and compressing the primary tableting mixture and secondary tableting mixture to provide the bilayer naproxen sodium tablet.

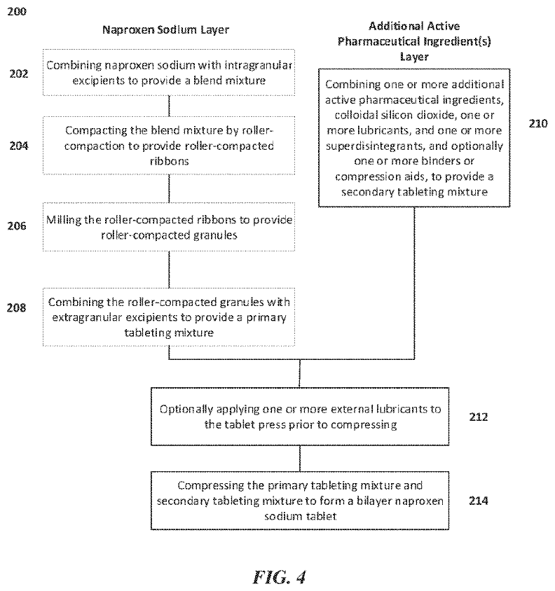

With reference to FIG. 4, process 200 is an exemplary process for preparing a bilayer naproxen sodium tablet as described herein. In step 202, naproxen sodium is combined with intragranular excipients (e.g., mannitol, colloidal silicon dioxide, stearic acid or magnesium stearate, and sodium starch glycolate) to form a blend mixture. The naproxen sodium and intragranular excipients are provided in dry powder forms. Similar to the process steps 104 and 106 described for process 100 above, the resulting blend mixture is processed in steps 204 and 206 to provide granulated naproxen sodium. The blend mixture comprising naproxen sodium and intragranular excipients are compacted by roller compaction to provide ribbons in step 204. In step 206, the ribbons are milled to provide roller-compacted granules. The roller-compacted granules are then combined with extragranular excipients (e.g., mannitol, sodium starch glycolate, starch and/or partially pregelatinized starch, stearic acid or magnesium stearate, croscarmellose sodium and optionally colloidal silicon dioxide) to provide a primary tableting mixture in step 208. In parallel step 210, one or more additional active pharmaceutical ingredients (such as acetaminophen) colloidal silicon dioxide, one or more lubricants, and one or more superdisintegrants, and optionally one or more binders or compression aids, are combined to provide a secondary tableting mixture. Following preparation of the two tableting mixtures, the two tableting mixtures are passed to a tablet press to be compressed. The tablet press may optionally be treated with external lubrication to facilitate the pressing of the tablet as provided in step 212. The primary tableting mixture and secondary tableting mixture are subsequently compressed to form the bilayer naproxen sodium tablet in step 214.

It should be recognized that the exemplary process 200 may be adapted to accommodate alternative active pharmaceutical ingredients, dissolution aids and/or excipients as described herein. It should also be understood that, in other variations, process 200 may include additional processing steps. In yet other variations, certain steps in process 200 may be omitted.

In still another aspect, provided herein is a method of preparing a naproxen sodium tablet, comprising: combining naproxen sodium, mannitol, colloidal silicon dioxide, stearic acid or magnesium stearate, and sodium starch glycolate to provide a blend mixture; compacting the blend mixture by roller compaction to provide ribbons; milling the ribbons to provide granules; combining the granules with mannitol, sodium starch glycolate, starch and/or partially pregelatinzed starch, stearic acid or magnesium stearate, croscarmellose sodium, and optionally colloidal silicon dioxide, to provide a primary tableting mixture; combining acetaminophen, colloidal silicon dioxide, starch and/or partially pregelatinized starch, stearic acid or magnesium stearate, and croscarmellose sodium to provide a secondary tableting mixture; and compressing the primary tableting mixture and secondary tableting mixture to provide the bilayer naproxen sodium tablet.

In some embodiments, the preparation of roller-compacted granules for the bilayer naproxen sodium tablet as described in steps 204 and 206 is similar to the preparation of granules as described for steps 104 and 106 in process 100 for a naproxen sodium tablet. Additionally, the characterization of roller-compacted granules for the bilayer naproxen sodium tablet as described in steps 204 and 206 is similar to the preparation of granules as described for steps 104 and 106 in process 100 for a naproxen sodium tablet.

For example, in some embodiments, the blend mixture as provided in the preparation of the bilayer naproxen sodium tablet may be compacted by roller compaction at an applied pressure, roller speed, and roller gap as described herein. In some embodiments which may be combined with the foregoing embodiments, the resulting roller-compacted ribbons may be characterized by their solid fraction, porosity, hardness and/or thickness as described herein. In still further embodiments, milling step to convert the ribbon material into granules may be characterized by mill speed. In still other embodiments, which may be combined with any of the preceding embodiments, the roller-compacted granules produced by the milling step may be characterized by their particle size distribution, bulk density and tapped density as described herein.

With further reference to FIG. 4, in step 212, external lubricants may be added to the tablet press or other tableting equipment prior to forming the bilayer tablet with the two tableting mixtures. The use of external lubricants may facilitate the ejection of the final tablet by reducing sticking of material to the tablet press. In still further embodiments of the foregoing, the method comprises optionally applying one or more external lubricants to the tablet press prior to compressing. In some embodiments, the one or more external lubricants comprise hypromellose, zinc stearate, carnauba wax, or any combinations thereof.

The process 200 for the preparation of the bilayer naproxen sodium tablet differs from the process 100 for preparation of a (monolayer) naproxen sodium tablet in the parallel preparation of secondary tableting mixture, as in step 210, and the details of the compression step 214. Depending upon the tablet press utilized, the compression of the two tableting mixtures in step 214 to form the bilayer naproxen sodium tablet may be carried out in a single compression step or in a two-step process comprising first (pre-)compressing one of the tableting mixtures to form one layer, subsequently loading the remaining tableting mixture into the press with the already prepared layer, and compressing the remaining tableting mixture and prepared layer to form the bilayer tablet. It should be recognized that, in instances wherein the two-step process for tableting is utilized, the sequence of compression may be ordered with either the primary tableting mixture or secondary tableting mixture being subjected to the pre-compression.

In some embodiments wherein the primary tableting mixture and secondary tableting mixture are compressed in a single compression step, the primary tableting mixture and secondary tableting mixture are compressed at a compression force of at least 1 kN, at least 2 kN, at least 3 kN, at least 4 kN, at least 5 kN, at least 10 kN, at least 15 kN, at least 20 kN, or at least 25 kN. In other embodiments, the primary tableting mixture and secondary tableting mixture are compressed at a compression force of less than or equal to 45 kN, less than or equal to 40 kN, less than or equal to 35 kN, less than or equal to 30 kN, less than or equal to 25 kN, or less than or equal to 20 kN. In certain embodiments, the primary tableting mixture and secondary tableting mixture are compressed at a compression force of between 5 kN and 45 kN, between 5 kN and 40 kN, between 5 kN and 35 kN, between 5 kN and 30 kN, between 5 kN and 25 kN, between 5 kN and 20 kN, between 5 kN and 15 kN, between 5 kN and 10 kN, between 10 kN and 45 kN, between 10 kN and 40 kN, between 10 kN and 35 kN, between 10 kN and 30 kN, between 10 kN and 25 kN, between 10 kN and 20 kN, between 10 kN and 15 kN, between 15 kN and 45 kN, between 15 kN and 40 kN, between 15 kN and 35 kN, between 15 kN and 30 kN, between 15 kN and 25 kN, between 15 kN and 20 kN, between 20 kN and 45 kN, between 20 kN and 40 kN, between 20 kN and 35 kN, between 20 kN and 30 kN, between 20 kN and 25 kN, between 25 kN and 45 kN, between 25 kN and 40 kN, between 25 kN and 35 kN, between 25 kN and 30 kN, between 30 kN and 45 kN, between 30 kN and 40 kN, between 30 kN and 35 kN, between 35 kN and 45 kN, between 35 kN and 40 kN, or between 40 kN and 45 kN.

In some embodiments wherein the primary tableting mixture and secondary tableting mixture are compressed in a two-step process, the primary tableting mixture or secondary tableting mixture may be compressed at a first compression force to form a first layer, followed by compression of the secondary tableting mixture or primary tableting mixture on top of the first layer at a second compression force to form the bilayer naproxen sodium tablet. As used herein, the terms “first layer” and “second layer” describe the order sequence in which a layer is prepared as part of the process to form a bilayer tablet; the term “primary layer” as used herein describe may be used to refer to the “naproxen sodium layer” and the term “secondary layer” may be used to refer to the layer of the bilayer tablet containing “one or more additional active pharmaceutical ingredients” such as acetaminophen.

In some embodiments, the method comprises compressing the primary tableting mixture to provide a first layer; and compressing the secondary tableting on top of the first layer to provide the bilayer naproxen sodium tablet. In certain embodiments, the method comprises compressing the primary tableting mixture to provide a naproxen sodium layer at a first compression force; and compressing the secondary tableting mixture on top of the naproxen sodium layer at a second compression force to provide the bilayer naproxen sodium tablet. In other embodiments, the method comprises compressing the secondary tableting mixture to provide a first layer; and compressing the primary tableting mixture on top of the first layer to provide the bilayer naproxen sodium tablet. In certain other embodiments, the method comprises compressing the secondary tableting mixture at a first compression force to provide an acetaminophen layer; and compressing the primary tableting mixture on top of the acetaminophen layer at a second compression force to provide the bilayer naproxen sodium tablet.

In some embodiments, the first compression force is at least 1 kN, at least 2 kN, at least 3 kN, at least 4 kN, at least 5 kN, at least 10 kN, at least 15 kN, at least 20 kN, or at least 25 kN. In other embodiments, the first compression force is less than or equal to 45 kN, less than or equal to 40 kN, less than or equal to 35 kN, less than or equal to 30 kN, less than or equal to 25 kN, or less than or equal to 20 kN. In some embodiments, the second compression force is at least 5 kN, at least 10 kN, at least 15 kN, at least 20 kN, or at least 25 kN. In other embodiments, the second compression force is less than or equal to 45 kN, less than or equal to 40 kN, less than or equal to 35 kN, less than or equal to 30 kN, less than or equal to 25 kN, or less than or equal to 20 kN.

In some embodiments, the first compression force and the second compression force are the same. In other embodiments, the first compression force and the second compression force are different. In still further embodiments, the first compression force is less than or equal to the second compression force.

### Methods of Use

In yet another aspect of the present disclosure, provided herein are methods of using the naproxen sodium tablets described herein.

As described herein, naproxen sodium may be used for the treatment of inflammation associated with a variety of conditions as well as for relief of mild to moderate pain.

In some embodiments, provided herein are methods of treating pain or ache in a subject in need thereof, comprising administering the naproxen sodium tablet to the subject. In certain embodiments of the foregoing embodiments, the pain or ache is associated with arthritis, headache, muscular ache, toothache, backache, the common cold, or menstrual cramps. In still other embodiments, provided herein is a method of reducing fever in a subject in need thereof, comprising administering the naproxen sodium tablet to the subject.

As described herein a subject may include but is not limited to a mammal, or more particularly a human.

In certain embodiments of the foregoing methods, the naproxen sodium tablet is administered orally. In still other embodiments, the naproxen sodium tablet is formulated for oral administration.

In other aspects, provided is an article of manufacture, such as a container comprising the naproxen sodium tablets as described herein, and a label containing instructions for use of the naproxen sodium tablets.

In yet other aspects, provided is a kit comprising the naproxen sodium tablets as described herein; and a package insert containing instructions for use of such naproxen sodium tablets.

In still yet another aspect, provided herein are methods of using the bilayer naproxen sodium tablets described herein. Similar to the naproxen tablets as described herein, the bilayer naproxen sodium tablets provided in the present disclosure may be used for the treatment of inflammation associated with a variety of conditions as well as for relief of mild to moderate pain.

In some embodiments, provided herein are methods of treating pain or ache in a subject in need thereof, comprising administering the bilayer naproxen sodium tablet to the subject. In certain embodiments of the foregoing embodiments, the pain or ache is associated with arthritis, headache, muscular ache, toothache, backache, the common cold, or menstrual cramps. In still other embodiments, provided herein is a method of reducing fever in a subject in need thereof, comprising administering the bilayer naproxen sodium tablet to the subject. In some embodiments of the foregoing methods, the administration step comprises administering two bilayer naproxen sodium tablets to the subject for a single dose.

In certain embodiments of the foregoing methods, the bilayer naproxen sodium tablet is administered orally. In still other embodiments, the bilayer naproxen sodium tablet is formulated for oral administration.

In other aspects, provided is an article of manufacture, such as a container comprising the bilayer naproxen sodium tablets as described herein, and a label containing instructions for use of the bilayer naproxen sodium tablets.

In yet other aspects, provided is a kit comprising the bilayer naproxen sodium tablets as described herein; and a package insert containing instructions for use of such bilayer naproxen sodium tablets.

### Enumerated Embodiments

The following enumerated embodiments are representative of some aspects of the invention.
1. A naproxen sodium tablet, comprising:

granules comprising naproxen sodium;

mannitol;

colloidal silicon dioxide;

one or more lubricants; and

one or more superdisintegrants,

wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.
2. A naproxen sodium tablet, comprising:

granules comprising naproxen sodium;

mannitol;

colloidal silicon dioxide;

stearic acid;

sodium starch glycolate; and

magnesium stearate,

wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.
3. The naproxen sodium tablet of embodiment 1 or embodiment 2, wherein the tablet comprises 60-80% w/w naproxen sodium.
4. The naproxen sodium tablet of any one of embodiments 1 to 3, wherein the naproxen sodium tablet comprises 10-20% w/w mannitol.
5. The naproxen sodium tablet of any one of embodiments 1 to 4, wherein the granules comprise mannitol, colloidal silicon dioxide, stearic acid and sodium starch glycolate.
6. The naproxen sodium tablet of any one of embodiments 1 to 5, wherein the granules are at least 85% w/w of the total weight of the naproxen sodium tablet.
7. The naproxen sodium tablet according to any one of embodiments 1 to 6, wherein the naproxen sodium tablet comprises mannitol, sodium starch glycolate, and magnesium stearate as extragranular excipients.
8. The naproxen sodium tablet according to any one of embodiments 1 to 7, wherein the naproxen sodium tablet comprises colloidal silicon dioxide as an extragranular excipient.
9. The naproxen sodium tablet according to any one of embodiments 1 to 8, further comprising a film coating.
10. The naproxen sodium tablet of any one of embodiments 1 to 9, wherein the naproxen sodium tablet has a disintegration time of less than 5 minutes as determined by the USP Disintegration Test in water using a basket-rack assembly with disks at 37° C.±0.5° C.
11. The naproxen sodium tablet according to any one of embodiments 1 to 10, wherein the naproxen sodium tablet has a hardness between 2 and 14 kilopond (kp) as determined by tablet tester in accordance with the USP Tablet Breaking Force Test.
12. The naproxen sodium tablet according to any one of embodiments 1 to 11, wherein the naproxen sodium tablet has a friability of less than or equal to 1% as determined by the USP Friability Test after 200 revolutions.
13. A method of preparing a naproxen sodium tablet according to embodiment 1, comprising:

combining naproxen sodium, mannitol, colloidal silicon dioxide, one or more lubricants and one or more superdisintegrants to provide a blend mixture;

compacting the blend mixture by roller compaction to provide ribbons;

milling the ribbons to provide granules;

combining the granules with mannitol, one or more lubricants, one or more superdisintegrants, and optionally colloidal silicon dioxide, to provide a tableting mixture; and

compressing the tableting mixture to provide the naproxen sodium tablet.
14. A method of preparing a naproxen sodium tablet according to any one of embodiments 1 to 12, comprising:

combining naproxen sodium, mannitol, colloidal silicon dioxide, stearic acid and sodium starch glycolate to provide a blend mixture;

compacting the blend mixture by roller compaction to provide ribbons;

milling the ribbons to provide granules;

combining the granules with mannitol, sodium starch glycolate, magnesium stearate, and optionally colloidal silicon dioxide, to provide a tableting mixture; and

compressing the tableting mixture to provide the naproxen sodium tablet.
15. The method of embodiments 13 or embodiment 14, wherein the blend mixture is compacted by roller compaction at an applied pressure between 18 and 30 bar.
16. The method of any one of embodiments 13 to 15, wherein the blend mixture is compacted by roller compaction at a roller speed between 4 and 9 rpm.
17. The method of any one of embodiments 13 to 16, wherein the blend mixture is compacted by roller compaction at a roller gap between 1.0 and 4.0 mm.
18. The method of any one of embodiments 13 to 17, wherein the ribbons have a porosity between 10% and 60%.
19. The method of any one of embodiments 13 to 18, wherein the ribbons have a solid fraction between 0.4 and 0.9.
20. The method of any one of embodiments 13 to 19, wherein the ribbons are milled to provide granules at a mill speed of between 40 and 160 rpm.
21. The method of any one of embodiments 13 to 20, wherein the granules have a particle size distribution wherein at least 50% w/w of the particles have a particle size greater than or equal to 250 μm.
22. The method of any one of embodiments 13 to 21, wherein the granules have a bulk density between 0.3 and 0.9 g/cc.
23. The method of any one of embodiments 13 to 22, wherein the granules have a tapped density between 0.6 and 0.9 g/cc.
24. The method of any one of embodiments 13 to 23, wherein the tableting mixture has a particle size distribution wherein at least 40% w/w of the particles have a particle size greater than or equal to 250 μm.
25. The method of claim any one of embodiments 13 to 24, wherein the tableting mixture has a bulk density between 0.3 and 0.9 g/cc.
26. The method of claim any one of embodiments 13 to 25, wherein the tableting mixture has a tapped density between 0.6 and 0.9 g/cc.
27. The method of any one of embodiments 13 to 26, wherein the tableting mixture is compressed with a compression force between 6 and 30 kN to provide the naproxen sodium tablet.
28. The method of any one of embodiments 13 to 27, further comprising coating the naproxen sodium tablet to provide a coated naproxen sodium tablet.
29. A naproxen sodium tablet obtained by the method of any one of embodiments 13 to 28.
30. A method of treating pain or ache in a subject in need thereof, comprising administering a naproxen sodium tablet according to any one of embodiments 1 to 12 or embodiment 29 to the subject.
31. The method of embodiment 30, wherein the pain or ache is associated with arthritis, muscular ache, backache, menstrual cramps, headache, toothache, or the common cold.
32. A method of reducing fever in a subject in need thereof, comprising administering a naproxen sodium tablet according to any one of embodiments 1 to 12 or embodiment 29 to the subject.
33. A bilayer naproxen sodium tablet, comprising:

a primary layer, comprising:
granules, comprising naproxen sodium;
mannitol;
colloidal silicon dioxide;
one or more binders;
one or more lubricants; and
one or more superdisintegrants, and

a secondary layer, comprising:
one or more additional active pharmaceutical ingredients;
colloidal silicon dioxide;
one or more binders;
one or more lubricants; and
one or more superdisintegrants,
   wherein the tablet has a disintegration time of less than 5 minutes as determined by the USP Disintegration Test in water using a basket-rack assembly with disks at 37° C.±0.5° C.
34. The bilayer naproxen sodium tablet of embodiment 33, wherein the one or more extragranular lubricants in the primary layer and the one or more lubricants in the secondary layer are the same.
35. The bilayer naproxen sodium tablet of embodiment 33 or embodiment 34, wherein the one or more extragranular superdisintegrants in the primary layer and the one or more superdisintegrants in the secondary layer are the same.
36. A bilayer naproxen sodium tablet, comprising:

a naproxen sodium layer, comprising:
granules, comprising naproxen sodium;
mannitol;
colloidal silicon dioxide;
sodium starch glycolate;
starch and/or partially pregelatinized starch;
stearic acid or magnesium stearate; and
croscarmellose sodium, and

an acetaminophen layer, comprising:
acetaminophen;
colloidal silicon dioxide;
starch and/or partially pregelatinized starch;
stearic acid or magnesium stearate, and
croscarmellose sodium,
   wherein the tablet has a disintegration time of less than 5 minutes as determined by the USP Disintegration Test in water using a basket-rack assembly with disks at 37° C.±0.5° C.
37. The bilayer naproxen sodium tablet of any one of embodiments 33 to 36, wherein the tablet comprises between 100 and 200 mg naproxen sodium.
38. The bilayer naproxen sodium tablet of any one of embodiments 33 to 37, wherein the naproxen sodium tablet comprises 150 mg naproxen sodium.
39. The bilayer naproxen sodium tablet of any one of embodiments 33 to 38, wherein the granules comprise mannitol, colloidal silicon dioxide, stearic acid or magnesium stearate, and sodium starch glycolate.
40. The bilayer naproxen sodium tablet of any one of embodiments 33 to 39, wherein the granules comprising naproxen sodium are at least 25% w/w of the total weight of the tablet.
41. The naproxen sodium tablet according to any one of embodiments 33 to 40, further comprising a film coating.
42. The naproxen sodium tablet of any one of embodiments 33 to 41, wherein the bilayer naproxen sodium tablet has a disintegration time of less than 4 minutes as determined by the USP Disintegration Test in water using a basket-rack assembly with disks at 37° C.±0.5° C.
43. The naproxen sodium tablet according to any one of embodiments 33 to 42, wherein the naproxen sodium tablet has a hardness between 2 and 14 kilopond (kp) as determined by tablet tester in accordance with the USP Tablet Breaking Force Test.
44. The naproxen sodium tablet according to any one of embodiments 33 to 43, wherein the naproxen sodium tablet has a friability of less than or equal to 1% as determined by the USP Friability Test after 200 revolutions.
45. A method of preparing a bilayer naproxen sodium tablet according to embodiment 33, comprising:

combining naproxen sodium, mannitol, colloidal silicon dioxide, one or more lubricants and one or more superdisintegrants to provide a blend mixture;

compacting the blend mixture by roller compaction to provide ribbons;

milling the ribbons to provide granules;

combining the granules with mannitol, one or more binders, one or more lubricants, one or more superdisintegrants, and optionally colloidal silicon dioxide, to provide a primary tableting mixture;

combining one or more additional active pharmaceutical ingredients, colloidal silicon dioxide, one or more lubricants, and one or more superdisintegrants to provide a secondary tableting mixture; and

compressing the primary tableting mixture and secondary tableting mixture to provide the bilayer naproxen sodium tablet.
46. A method of preparing a naproxen sodium tablet according to any one of embodiments 33 to 44, comprising:

combining naproxen sodium, mannitol, colloidal silicon dioxide, stearic acid or magnesium stearate, and sodium starch glycolate to provide a blend mixture;

compacting the blend mixture by roller compaction to provide ribbons;

milling the ribbons to provide granules;

combining the granules with mannitol, sodium starch glycolate, starch and/or partially pregelatinzed starch, stearic acid or magnesium stearate, croscarmellose sodium, and optionally colloidal silicon dioxide, to provide a primary tableting mixture; and

combining acetaminophen, colloidal silicon dioxide, starch and/or partially pregelatinized starch, stearic acid or magnesium stearate, and croscarmellose sodium to provide a secondary tableting mixture; and

compressing the primary tableting mixture and secondary tableting mixture to provide the bilayer naproxen sodium tablet.
47. The method of embodiments 45 or embodiment 46, wherein the blend mixture is compacted by roller compaction at an applied pressure between 18 and 30 bar.
48. The method of any one of embodiments 45 to 47, wherein the blend mixture is compacted by roller compaction at a roller speed between 4 and 9 rpm.
49. The method of any one of embodiments 45 to 48, wherein the blend mixture is compacted by roller compaction at a roller gap between 1.0 and 4.0 mm.
50. The method of any one of embodiments 45 to 49, wherein the ribbons have a porosity between 10% and 60%.
51. The method of any one of embodiments 45 to 50, wherein the ribbons have a solid fraction between 0.4 and 0.9.
52. The method of any one of embodiments 45 to 51, wherein the ribbons are milled to provide granules at a mill speed of between 40 and 160 rpm.
53. The method of any one of embodiments 45 to 52, wherein the granules have a particle size distribution wherein at least 50% w/w of the particles have a particle size greater than or equal to 250 μm.
54. The method of any one of embodiments 45 to 53, wherein the granules have a bulk density between 0.3 and 0.9 g/cc.
55. The method of any one of embodiments 45 to 54, wherein the granules have a tapped density between 0.6 and 0.9 g/cc.
56. The method of any one of embodiments 45 to 55, wherein the tableting mixture has a particle size distribution wherein at least 40% w/w of the particles have a particle size greater than or equal to 250 μm.
57. The method of claim any one of embodiments 45 to 56, wherein the tableting mixture has a bulk density between 0.3 and 0.9 g/cc.
58. The method of claim any one of embodiments 45 to 57, wherein the tableting mixture has a tapped density between 0.6 and 0.9 g/cc.
59. The method of any one of embodiments 45 to 58, wherein the primary tableting mixture and secondary tableting mixture are compressed with a compression force between 6 and 30 kN to provide the bilayer naproxen sodium tablet.
60. The method of any one of embodiments 45 to 59, wherein compressing the primary tableting mixture and secondary tableting mixture to form the bilayer naproxen sodium tablet comprises:

compressing the primary tableting mixture at a first compression force to provide an naproxen sodium layer; and

compressing the secondary tableting mixture on top of the naproxen sodium layer at a second compression force to form the bilayer naproxen sodium tablet.
61. The method of any one of embodiments 45 to 59, wherein compressing the primary tableting mixture and secondary tableting mixture to form the bilayer naproxen sodium tablet comprises:

compressing the secondary tableting mixture at a first compression force to provide a first layer; and

compressing the primary tableting mixture blend on top of the first layer at a second compression force to form the bilayer naproxen sodium tablet.
62. The method of embodiment 60 or embodiment 61, wherein the first compression force is between 1 kN and 30 kN and the second compression force is between 5 kN and 30 kN.
63. The method of any one of embodiments 45 to 62, further comprising coating the bilayer naproxen sodium tablet to provide a coated bilayer naproxen sodium tablet.
64. A bilayer naproxen sodium tablet obtained by the method of any one of embodiments 45 to 63.
65. A method of treating pain or ache in a subject in need thereof, comprising administering a bilayer naproxen sodium tablet according to any one of embodiments 33 to 44 or embodiment 64 to the subject.
66. The method of embodiment 65, wherein the pain or ache is associated with arthritis, muscular ache, backache, menstrual cramps, headache, toothache, or the common cold.
67. A method of reducing fever in a subject in need thereof, comprising administering a bilayer naproxen sodium tablet according to any one of embodiments 33 to 44 or embodiment 64 to the subject.

### EXAMPLES

The presently disclosed subject matter will be better understood by reference to the following Examples, which are provided as exemplary of the invention, and not by way of limitation.

### Example 1

### Preparation of Naproxen Sodium Tablet

The present example describes the preparation of a naproxen sodium tablet by dry granulation employing mannitol, dibasic calcium phosphate or both, as intragranular and extragranular diluents. Table 1A shows three trial blend mixtures used for roller compaction in the present example. The three trial blend mixtures were combined in the mass proportions shown in Table 1A, passed through roller compactors (at roller speed of 9 rpm, a roller pressure of 20 bar, and a roller gap of 4.0mm), and the resulting compacted ribbons milled (at a mill speed of 107 rpm).

TABLE 1A

| Blend Mixture | Trial 1 | Trial 2 | Trial 3 |
|---|---|---|---|
| (Roller-Compacted Granules) | (mg/tablet) | (mg/tablet) | (mg/tablet) |
| naproxen sodium | 220 | 220 | 220 |
| mannitol | 50 | — | 25 |
| dibasic calcium phosphate | — | 50 | 25 |
| colloidal silicon dioxide | 3 | 3 | 3 |
| stearic acid | 3 | 3 | 3 |
| sodium starch glycolate | 6 | 6 | 6 |
| Granule sub-total | 282 | 282 | 282 |

The roller-compacted granules produced from each of the three trails were further combined with the corresponding extragranular excipients in the mass proportions as shown in Table 1B, and compressed into tablets (compression force 15 kN).

TABLE 1B

| Blend Mixture | Trial 1 | Trial 2 | Trial 3 |
|---|---|---|---|
| (Roller-Compacted Granules) | (mg/tablet) | (mg/tablet) | (mg/tablet) |
| roller-compacted granules | 282 | 282 | 282 |
| mannitol | 10 | — | 5 |
| dibasic calcium phosphate | — | 10 | 5 |
| sodium starch glycolate | 8 | 8 | 8 |
| magnesium stearate | 5 | 5 | 5 |
| Core Tablet Weight | 305 | 305 | 305 |

The naproxen sodium tablets prepared as described above were tested for their dissolution profile in comparison to a commercially available naproxen sodium tablet (“standard”) prepared by fluid bed granulation.

The commercially available naproxen sodium tablet (“Comparative Example”) was prepared with the ingredients shown in Table 2. Commercially available naproxen sodium tablets may be prepared in accordance with the general procedure detailed below. Naproxen sodium is first combined with microcrystalline cellulose, povidone and water to provide granules via wet granulation method (high shear/fluid bed). The granules are then dried to a certain moisture content, and milled to a certain particle size. The milled granules are further combined with microcrystalline cellulose, talc and magnesium stearate to facilitate compression and ejection in the next tableting step. The mixture is tableted and coated with a suitable tablet coating to provide the final naproxen sodium tablet.

TABLE 2

| Comparative Example - | | |
|---|---|---|
| Commercial Naproxen | | |
| Sodium | mg/tablet | % |
| naproxen sodium | 220 | 72.57 |
| microcrystalline cellulose | 22.01 | 7.26 |
| Povidone (K29-32) | 10 | 3.30 |
| purified water* | 14.44 | 4.76 |
| Wet Granules Weight | 266.44 | 87.89 |
| microcrystalline cellulose | 22 | 7.26 |
| talc | 12.6 | 4.16 |
| magnesium stearate | 2.1 | 0.64 |
| Core Tablet Weight | 303.15 | 100.00 |
| Opadry Blue YS-1-4215 | 6.06-9.09 | — |
| Total Tablet Weight | 310.72 | — |

*water remains in the granulation after drying

The dissolution profiles of the test tablets from the three trials and the Comparative Example tablet were determined in accordance with the U.S. Pharmacopeia standardized protocol for dissolution of immediate-release dosage forms of naproxen sodium (USP34-NF29 Chapter <711> Dissolution, Stage 6 Harmonization Bulletin dated Dec. 1, 2011; and Naproxen Sodium monograph USP41-NF36, Interim Revision Announcement dated May 1, 2018), which is briefly summarized here.

A single tablet is placed in a paddle apparatus (Apparatus 2) containing 0.1 M phosphate buffer of pH 7.4 (900 mL, equilibrated to 37±0.5° C.), at paddle rotation speed of 50 rpm. Aliquots of the phosphate buffer were taken at 10 minutes, 20 minutes, 30 minutes and 45 minutes. The quantity of naproxen sodium dissolved in the dissolution medium was determined by UV absorption spectrometry at 332 nm. The dissolution measurements were taken for six tablets for each trial.

Table 3 shows the observed percentage of naproxen dissolved in phosphate buffer pH 7.4 at each time point, as the average of six measurements for each trial. The formulation comprising mannitol as the primary diluent was found to have the fastest dissolution overall.

TABLE 3
Percentage of naproxen sodium dissolved
in phosphate buffer pH 7.4

| Time | | | | Comparative |
|---|---|---|---|---|
| (minutes) | Trial 1 | Trial 2 | Trial 3 | Example Tablet |
| 0 | 0 | 0 | 0 | 0 |
| 10 | 83.4 | 67.7 | 73.4 | 64 |
| 20 | 98.2 | 91.8 | 96.1 | 93 |
| 30 | 98.1 | 96.9 | 98.4 | 97 |
| 45 | 98.1 | 99.9 | 100.5 | 98 |
| 60 | — | — | — | 99 |

### Example 2

### Dissolution Profile of Naproxen Sodium Tablets in Variable Dissolution Media

The present example describes the preparation of a naproxen sodium tablet by dry granulation and evaluation of its dissolution profile in different dissolution media.

Naproxen sodium, USP grade, was combined with mannitol (Mannogem EZ, Spray Dried), colloidal silicon dioxide (Cab-O-Sil®), stearic acid and sodium starch glycolate (Explotab®) in the proportions detailed in Table 4A (identical to Trial 1 in Table 1A) to prepare a blend mixture for subsequent roller compaction.

The blend mixture was passed through a roller compactor (roller speed9 rpm; roller pressure 20 bar; roller gap 4.0 mm) to provide roller-compacted ribbons, which were then milled (mill speed 107 rpm) through screens of certain openings to provide free-flowing roller-compacted granules containing naproxen sodium.

TABLE 4A

| Blend Mixture (Roller- | | |
|---|---|---|
| Compacted Granules) | mg/tablet | %* |
| naproxen sodium | 220 | 72.13 |
| mannitol | 50 | 16.39 |
| colloidal silicon dioxide | 3 | 0.98 |
| stearic acid | 3 | 0.98 |
| sodium starch glycolate | 6 | 1.97 |
| Granule Sub-Total | 282 | 92.46%* |

*by weight of core tablet (Table 2)

The roller-compacted granules were then mixed with additional excipients mannitol (Pearlitol® SD200, spray-dried), sodium starch glycolate (Explotab), and magnesium stearate in the proportions shown in Table 4B (identical to Trial 1 in Table 1B). The resulting final blend (tableting mixture) was compressed in a tablet press at 15 kN compression force to provide uncoated naproxen sodium tablets.

TABLE 4B

| Core Tablet | mg/tablet | % |
|---|---|---|
| roller-compacted granules | 282 | 92.46 |
| mannitol | 10 | 3.28 |
| sodium starch glycolate | 8 | 2.62 |
| magnesium stearate | 5 | 1.64 |
| Core Tablet Weight | 305 | 100.00 |

The uncoated tablets produced by the tableting step were then coated with one of two film coatings (Opadry® YS-1-4215 and Opadry® QX).

The dissolution profiles of the coated test tablets and the Comparative Example tablet provided in Example 1 (Table 2) were determined in accordance with the USP Dissolution Test as described in Example 1 above. A single tablet is placed in a paddle apparatus (Apparatus 2) containing 0.1 M phosphate buffer of pH 7.4 (900 mL, equilibrated to 37±0.5° C.), at paddle rotation speed of 50 rpm. Aliquots of the phosphate buffer were taken at 10 minutes, 20 minutes, 30 minutes and 45 minutes. The quantity of naproxen sodium dissolved in the dissolution medium was determined by UV absorption spectrometry at 332 nm.

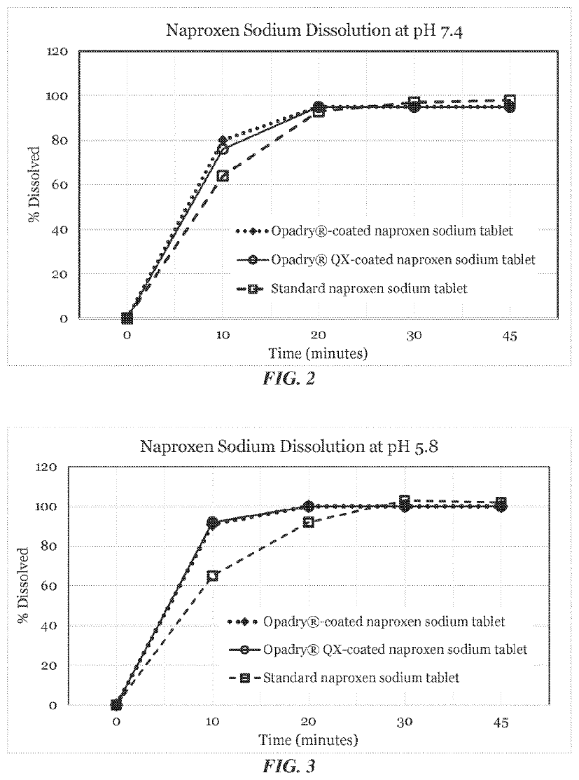

Table 5 shows the observed percentage of naproxen dissolved in phosphate buffer pH 7.4 at each time point, as the average of six tablets for each trial. FIG. 2 shows a comparative plot of the dissolution profiles of the naproxen sodium tablets (that is, the percent of naproxen sodium in solution as a percentage of the total naproxen sodium in the starting tablet) as a function of time in phosphate buffer at pH 7.4.

TABLE 5

| | Percentage of naproxen sodium | | |
|---|---|---|---|
| | dissolved in phosphate buffer pH 7.4 | | |
| | Opadry ®- | Opadry ® | Comparative |
| Time | coated naproxen | QX-coated naproxen | Example |
| (minutes) | sodium tablet | sodium tablet | Tablet |
| 0 | 0 | 0 | 0 |
| 10 | 80 | 76 | 64 |
| 20 | 95 | 95 | 93 |
| 30 | 95 | 95 | 97 |
| 45 | 95 | 95 | 98 |

Additional assessments to determine the dissolution profiles of coated naproxen sodium tablets prepared by dry granulation/roller compaction under acidic conditions were also carried out. The USP dissolution protocol for naproxen sodium immediate-release tablets was adapted to substitute the dissolution media with phosphate buffer at pH 5.8 in lieu of the standard phosphate buffer at pH 7.4. Table 6 shows the observed percentage of naproxen dissolved in phosphate buffer pH 5.8 at each time point. FIG. 3 shows a comparative plot of the dissolution profiles of the naproxen sodium tablets as a function of time in pH 5.8 buffer as compared to the same tablets in pH 7.4 buffer. The results in Table 6 are the average percentages for six tablets for each trial.

TABLE 6

| | Percentage of naproxen sodium | | |
|---|---|---|---|
| | dissolved in phosphate buffer pH 5.8 | | |
| | Opadry ®- | Opadry ® | Comparative |
| Time | coated naproxen | QX-coated naproxen | Example |
| (minutes) | sodium tablet | sodium tablet | Tablet |
| 0 | 0 | 0 | 0 |
| 10 | 91 | 92 | 65 |
| 20 | 100 | 100 | 92 |
| 30 | 100 | 100 | 103 |
| 45 | 100 | 100 | 102 |

As shown in FIG. 2, the naproxen sodium tablets prepared by dry granulation demonstrated a superior dissolution profile to that of naproxen sodium tablets prepared by wet granulation. As further shown in FIG. 3, the naproxen sodium tablets prepared by dry granulation/roller compaction exhibited the same dissolution profile in an acidic medium.

### Example 3

### Process Parameter Evaluation

The present example describes the evaluation of the effects of various parameters in the dry granulation/roller compaction process on the properties of the resulting processed material (roller-compacted ribbons, granules, and blend and tableting mixtures) as well as the dissolution profile, disintegration time, hardness and friability of the final naproxen sodium tablets.

As shown in this Example, the formulation for the naproxen sodium tablets as described herein results in process material that exhibit consistent physical properties and final naproxen sodium tablets demonstrating consistently enhanced dissolution even when initially subjected to variable roller compaction parameters.

### Part I—Roller Compaction Parameters

Blend mixtures of naproxen sodium and intragranular excipients (mannitol, colloidal silicon dioxide, stearic acid, sodium starch glycolate) for the roller-compacted granules were prepared in accordance with the mass proportions detailed in Table 4A above. The blend mixtures were subjected to thirteen different runs under varied roller compaction process conditions as shown in Table 7 below.

TABLE 7

| | Roller Speed | Roller Gap | Roller Pressure | |
|---|---|---|---|---|
| Run # | (rpm) | (mm) | (bar) | Roller Type |
| 1 | 7 | 3 | 20 | SER/SER |
| 2 | 9 | 4 | 18 | SM/SER |
| 3 | 4 | 4 | 18 | SER/SER |
| 4 | 9 | 1 | 30 | SM/SER |
| 5 | 7 | 3 | 20 | SM/SER |
| 6 | 9 | 4 | 30 | SER/SER |
| 7 | 7 | 3 | 20 | SER/SER |
| 8 | 4 | 1 | 18 | SM/SER |
| 9 | 9 | 1 | 18 | SER/SER |
| 10 | 7 | 3 | 20 | SM/SER |
| 11 | 4 | 1 | 30 | SER/SER |
| 12 | 4 | 4 | 30 | SM/SER |
| 13 | 9 | 4 | 20 | SER/SER |

Each of the thirteen runs employed a different combination of values for roller speed, roller gap, roller pressure, and roller type in order to evaluate their aggregate effect on the resulting roller-compacted ribbons. The resulting roller-compacted ribbons were evaluated for their hardness, thickness, true density, envelope density, solid fraction, and porosity. The results are shown in Table 8.

As shown in Table 8, the roller-compacted ribbons produced under a range of roller compaction parameters showed fairly constant porosity.

TABLE 8
Roller-Compacted Ribbons

| | | True | Envelope | | |
|---|---|---|---|---|---|
| | Hardness | Density | Density | Porosity | Thickness |
| Run # | (g) | (g/cc) | (g/cc) | (%) | (mm) |
| 1 | 105.62 | 1.395 | 0.9865 | 29.3 | 1.7 |
| 2 | 113.00 | 1.701 | 1.077 | 22.8 | 1.5 |
| 3 | 117.38 | 1.391 | 0.986 | 29.1 | 2.6 |
| 4 | 82.61 | 1.375 | 1.064 | 22.6 | 1.1 |
| 5 | 119.87 | 1.395 | 0.961 | 31.1 | 1.7 |
| 6 | 99.84 | 1.381 | 1.005 | 27.2 | 1.4 |
| 7 | 91.80 | 1.390 | 0.997 | 28.7 | 1.6 |
| 8 | 131.90 | 1.232 | 0.959 | 22.2 | 1.6 |
| 9 | 78.50 | 1.381 | 1.026 | 25.7 | 1.2 |
| 10 | 102.24 | 1.391 | 0.973 | 30.1 | 1.7 |
| 11 | 238.27 | 1.398 | 0.987 | 29.4 | 1.6 |
| 12 | 935.85 | 1.399 | 1.025 | 26.8 | 3.9 |
| 13 | 90.26 | 1.391 | 0.935 | 32.8 | 1.4 |

### Part II—Mill Speed

Prior to carrying out milling of the ribbons prepared in Part I above, a brief evaluation of the effect of mill speed on granule particle size was carried out. A blend mixture was prepared in accordance with the mass proportions in Table 4A above. The blend mixture was compacted with roller compactors at a feed rate of 80%, roller speed of 7 rpm, roller pressure of 20 bar, and a roller gap of 2.0 mm. The resulting ribbons were passed through a mill at one of three different mill speeds (60 rpm, 85 rpm, and 108 rpm) to provide granules.

The resulting granules were passed sequentially through seven sieves of mesh sizes (and corresponding nominal sieve opening): No. 20 (841 μm), No. 40 (420 μm), No. 60 (250 μm), No. 80 (177 μm), No. 100 (149 μm), No. 200 (74 μm) and No. 325 (44 μm), and the mass of material retained on each sieve recorded. The total mass retained on each sieve was calculated as a percentage of the total mass of material passed through the series of seven sieves to determine the particle size distribution of the roller-compacted granules. The particle size distributions of the granules obtained from the three different mill speeds are shown in Table 9.

TABLE 9
Roller-Compacted Granules-Particle Size Distribution (%)

| | No. 20 | No. 40 | No. 60 | No. 80 | No. 100 | No. 200 | No. 325 | Pan |
|---|---|---|---|---|---|---|---|---|
| Mill | (>841 | (>420 | (>250 | (>177 | (>149 | (>75 | (>45 | (≤44 |
| Speed | μm) | μm) | μm) | μm) | μm) | μm) | μm) | μm) |
| 60 rpm | 26.4 | 28.8 | 12.3 | 6.1 | 2.6 | 10.1 | 3.9 | 9.7 |
| 85 rpm | 28.6 | 27.1 | 11.4 | 6.3 | 2.6 | 10.6 | 4.4 | 9.1 |
| 108 rpm | 30.0 | 28.0 | 11.7 | 6.6 | 2.4 | 10.0 | 3.7 | 7.6 |

The resulting granules produced at the three different mill speeds showed little variation in the resulting granule size distribution.

### Part III—Granule and Tableting Blend Particle Size Distributions

Following assessment of the ribbon properties, the ribbons from each of the thirteen runs in Part I were milled (mill speed 85 rpm) into granules, and the granules sieved to determine particle size distribution. The granules were passed sequentially through seven sieves of mesh sizes (and corresponding nominal sieve opening): No. 20 (841 μm), No. 40 (420 μm), No. 60 (250 μm), No. 80 (177 μm), No. 100 (149 μm), No. 200 (74 μm) and No. 325 (44 μm), and the mass of material retained on each sieve recorded. The total mass retained on each sieve was calculated as a percentage of the total mass of material passed through the series of seven sieves to determine the particle size distribution of the roller-compacted granules. The particle size distributions of the granules obtained from the thirteen runs are shown in Table 10.

TABLE 10
Roller-Compacted Granules-Particle Size Distribution (%)

| | No. 20 | No. 40 | No. 60 | No. 80 | No. 100 | No. 200 | No. 325 | Pan |
|---|---|---|---|---|---|---|---|---|
| Run | (>841 | (>420 | (>250 | (>177 | (>149 | (>75 | (>45 | (≤44 |
| # | μm) | μm) | μm) | μm) | μm) | μm) | μm) | μm) |
| 1 | 16.7 | 34.5 | 13.7 | 6.3 | 2.8 | 10.5 | 6.6 | 8.9 |
| 2 | 13.2 | 33.2 | 15.9 | 7.2 | 3.0 | 11.3 | 7.1 | 9.0 |
| 3 | 14.8 | 33.7 | 13.4 | 6.3 | 3.0 | 11.3 | 7.0 | 10.4 |
| 4 | 19.1 | 39.1 | 13.3 | 5.5 | 2.5 | 8.9 | 5.1 | 6.5 |
| 5 | 20.6 | 37.8 | 12.6 | 5.3 | 2.4 | 8.2 | 5.1 | 8.0 |
| 6 | 14.1 | 34.9 | 14.9 | 7.1 | 3.2 | 10.6 | 6.5 | 8.7 |
| 7 | 15.4 | 33.2 | 13.2 | 6.3 | 2.9 | 10.4 | 7.1 | 10.7 |
| 8 | 17.3 | 37.7 | 13.4 | 6.2 | 2.7 | 9.4 | 5.6 | 6.8 |
| 9 | 16.5 | 37.6 | 13.1 | 5.8 | 2.7 | 9.8 | 6.3 | 7.4 |
| 10 | 15.5 | 32.6 | 13.6 | 7 | 2.7 | 10.9 | 6.6 | 10.3 |
| 11 | 15.3 | 36.5 | 14.0 | 6.5 | 2.9 | 10.1 | 6.6 | 6.9 |
| 12 | 9.3 | 30.3 | 17.6 | 9.5 | 3.9 | 12.0 | 7.8 | 7.9 |
| 13 | 16.0 | 34.2 | 13.0 | 6.1 | 3.3 | 10.3 | 6.8 | 9.1 |

The granules obtained from the thirteen runs were then mixed with extragranular excipients (mannitol, sodium starch glycolate, magnesium stearate, and colloidal silicon dioxide) to provide tableting mixtures (according to the proportions of Table 11).

TABLE 11

| Core Tablet | mg/tablet | % |
|---|---|---|
| roller-compacted granules | 282 | 91.6% |
| mannitol | 10 | 3.2% |
| sodium starch glycolate | 8 | 2.6% |
| magnesium stearate | 5 | 1.6% |
| colloidal silicon dioxide | 3 | 1.0% |
| Core Tablet Weight | 308 | 100.00 |

Prior to tableting, however, the tableting blends were sieved in order to determine their particle size distributions. Similar to the analysis for the granules above, the tableting blends for each run were passed sequentially through seven sieves of mesh sizes (and corresponding nominal sieve opening): No. 20 (841 μm), No. 40 (420 μm), No. 60 (250 μm), No. 80 (177 μm), No. 100 (149 μm), No. 200 (74 μm) and No. 325 (44 μm), and the mass of material retained on each sieve recorded. The total mass retained on each sieve was calculated as a percentage of the total mass of material passed through the series of seven sieves to determine the particle size distribution of the final tableting blends, which are shown in Table 12. The particle size distribution of the final tableting blends was fairly similar to the particle size distribution of the roller-compacted granules.

TABLE 12
Final Tableting Blend-Particle Size Distribution (%)

| | No. 20 | No. 40 | No. 60 | No. 80 | No. 100 | No. 200 | No. 325 | Pan |
|---|---|---|---|---|---|---|---|---|
| Run | (>841 | (>420 | (>250 | (>177 | (>149 | (>75 | (>45 | (≤44 |
| # | μm) | μm) | μm) | μm) | μm) | μm) | μm) | μm) |
| 1 | 12.8 | 28.5 | 13.4 | 6.5 | 3.5 | 13.3 | 7.6 | 14.4 |
| 2 | 12.9 | 27.3 | 12.5 | 6.6 | 3.5 | 13.4 | 9.1 | 14.8 |
| 3 | 12.0 | 26.3 | 12.9 | 7.1 | 3.0 | 14.8 | 8.7 | 15.1 |
| 4 | 14.2 | 34.2 | 13.1 | 5.8 | 3.0 | 11.6 | 7.1 | 11.0 |
| 5 | 12.6 | 29.6 | 12.7 | 6.9 | 3.9 | 13.3 | 8.1 | 12.9 |
| 6 | 11.5 | 31.3 | 14.5 | 6.6 | 3.2 | 12.2 | 7.7 | 12.9 |
| 7 | 12.7 | 28.6 | 13.2 | 6.4 | 3.3 | 13.0 | 9.1 | 13.3 |
| 8 | 11.6 | 30.9 | 13.4 | 6.4 | 3.2 | 12.8 | 8.7 | 12.9 |
| 9 | 11.3 | 29.7 | 13.4 | 6.1 | 3.2 | 12.9 | 8.4 | 12.5 |
| 10 | 10.8 | 27.9 | 13.5 | 6.8 | 3.4 | 13.4 | 8.6 | 14.0 |
| 11 | 12.9 | 35.8 | 13.7 | 6.2 | 3.2 | 11.1 | 7.0 | 9.0 |
| 12 | 13.3 | 33.4 | 13.5 | 6.2 | 3.6 | 11.2 | 7.3 | 10.5 |
| 13 | 10.0 | 26.3 | 11.5 | 7.3 | 3.9 | 13.9 | 10.1 | 14.5 |

The bulk density, tapped density and Carr's index (compressibility) of both the roller-compacted granules and the tableting mixtures (final blends) were also determined, as shown in Table 13.

TABLE 13

| | Roller-Compacted Granules | | | Final Tableting Blend | | |
|---|---|---|---|---|---|---|
| | Bulk | Tapped | Carr's | Bulk | Tapped | Carr's |
| | Density | Density | Index | Density | Density | Index |
| Run # | (g/cc) | (g/cc) | % | (g/cc) | (g/cc) | % |
| 1 | 0.5902 | 0.7567 | 22% | 0.5527 | 0.7370 | 25% |
| 2 | 0.5749 | 0.7371 | 22% | 0.5373 | 0.7463 | 28% |
| 3 | 0.5738 | 0.7549 | 24% | 0.5422 | 0.7325 | 26% |
| 4 | 0.6187 | 0.7639 | 19% | 0.5753 | 0.7774 | 26% |
| 5 | 0.5926 | 0.7408 | 20% | 0.5584 | 0.7546 | 26% |
| 6 | 0.5874 | 0.7436 | 21% | 0.5611 | 0.7686 | 27% |
| 7 | 0.5752 | 0.7374 | 22% | 0.5467 | 0.7488 | 27% |
| 8 | 0.5946 | 0.7527 | 21% | 0.5767 | 0.7689 | 25% |
| 9 | 0.6031 | 0.7634 | 21% | 0.5711 | 0.7615 | 25% |
| 10 | 0.5810 | 0.7546 | 23% | 0.5466 | 0.7487 | 27% |
| 11 | 0.6200 | 0.7750 | 20% | 0.5958 | 0.7839 | 24% |
| 12 | 0.5765 | 0.7391 | 22% | 0.5874 | 0.7728 | 24% |
| 13 | 0.5857 | 0.7606 | 23% | 0.5460 | 0.7479 | 27% |

### Part III—Tableting

The thirteen tableting blends in Part II above were each compressed in a tablet press under six different compression forces (6, 10.8, 15.5, 20.8, 25, and 30 kN). The final weight, thickness and hardness of the tablets obtained for the thirteen blends under the six compression forces were measured using a semi-automated tablet tester (Sotax Pharmatest ST50). The thickness and hardness of the tablets compressed under various compression forces is shown in Tables 14-15 below.

TABLE 14
Thickness

| | Compression Force (kN) | | | | | |
|---|---|---|---|---|---|---|
| Run # | 6 kN | 10.8 kN | 15.5 kN | 20.8 kN | 25 kN | 30 kN |
| 1 | 5.08 | 4.94 | 4.79 | 4.76 | 4.72 | 4.71 |
| 2 | 5.22 | 5.01 | 4.91 | 4.87 | 4.85 | 4.77 |
| 3 | 5.18 | 4.94 | 4.83 | 4.75 | 4.72 | 4.72 |
| 4 | 5.38 | 5.2 | 5.11 | 5.01 | 4.98 | n/a |
| 5 | 5.17 | 5 | 4.9 | 4.84 | 4.81 | 4.81 |
| 6 | 5.26 | 5.06 | 4.88 | 4.78 | 4.76 | 4.74 |
| 7 | 5.22 | 5.02 | 4.9 | 4.83 | 4.82 | 4.77 |
| 8 | 5.19 | 4.96 | 4.86 | 4.81 | 4.77 | 4.76 |
| 9 | 5.17 | 5.03 | 4.95 | 4.88 | 4.84 | 4.82 |
| 10 | 5.17 | 5.07 | 4.93 | 4.89 | 4.84 | 4.83 |
| 11 | 5.25 | 5.11 | 4.97 | 4.87 | 4.83 | 4.82 |
| 12 | 5.24 | 5.1 | 4.95 | 4.91 | 4.88 | 4.85 |
| 13 | 5.24 | 5 | 4.85 | 4.75 | 4.72 | 4.68 |

TABLE 15
Hardness

| | Compression Force (kN) | | | | | |
|---|---|---|---|---|---|---|
| Run # | 6 kN | 10.8 kN | 15.5 kN | 20.8 kN | 25 kN | 30 kN |
| 1 | 4.9 | 7.4 | 9.3 | 11.1 | 11.4 | 11.64 |
| 2 | 4.8 | 7.7 | 9.8 | 9.8 | 10.2 | 6.4 |
| 3 | 5.1 | 7.8 | 9.9 | 10.625 | 10.5 | 7.8 |
| 4 | 4 | 7.5 | 9.7 | 10.6 | 10.5 | n/a |
| 5 | 5.3 | 8 | 7.9 | 9.9 | 9.26 | 3.3 |
| 6 | 5.1 | 7.6 | 9.2 | 10.09 | 9.7 | 6 |
| 7 | 4.7 | 6.3 | 9.6 | 10.8 | 10.4 | 6.6 |
| 8 | 2.02 | 7.8 | 9.6 | 9.9 | 10.43 | 6.6 |
| 9 | 4.7 | 6.9 | 8.3 | 10.6 | 10.6 | 6 |
| 10 | 5.5 | 7.7 | 9.8 | 9.7 | 9.6 | 4.9 |
| 11 | 4.6 | 4.25 | 9.4 | 10.9 | 9.1 | 5.5 |
| 12 | 3.1 | 8 | 9.6 | 10.44 | 8.6 | 5 |
| 13 | 4.8 | 6.04 | 9.09 | 10.5 | 10.06 | 4.4 |

Tablet disintegration was also assessed for the 78 tablet runs (thirteen roller-compaction runs×compression forces), in accordance with USP disintegration test (USP43-NF38, Chapter <701> Disintegration, Stage 4 Harmonization Bulletin dated Apr. 26, 2019; uncoated tablet procedure, basket-rack assembly) in water in a basket-rack assembly with disks. The results are shown in Table 16 below.

TABLE 16
Tablet Disintegration (min:sec)

| | Compression Force (kN) | | | | | |
|---|---|---|---|---|---|---|
| Run # | 6 kN | 10.8 kN | 15.5 kN | 20.8 kN | 25 kN | 30 kN |
| 1 | 03:44 | 03:44 | 03:57 | 04:08 | 04:13 | 04:01 |
| 2 | 04:04 | 04:14 | 04:05 | 04:02 | 04:13 | 04:13 |
| 3 | 04:12 | 03:23 | 03:43 | 03:20 | 03:34 | 04:06 |
| 4 | 04:44 | 04:8 | 04:06 | 04:15 | 04:14 | n/a |
| 5 | 04:01 | 03:53 | 04:02 | 03:44 | 04:10 | 04:09 |
| 6 | 04:20 | 04:08 | 03:58 | 04:03 | 04:12 | 04:15 |
| 7 | 04:01 | 04:08 | 04:03 | 04:21 | 03:51 | 03:56 |
| 8 | 04:32 | 04:09 | 03:50 | 04:16 | 03:38 | 04:02 |
| 9 | 03:54 | 03:57 | 04:05 | 03:46 | 03:36 | 03:30 |
| 10 | 03:58 | 03:55 | 03:50 | 03:43 | 03:58 | 03:46 |
| 11 | 04:05 | 03:35 | 03:47 | 04:03 | 03:46 | 04:03 |
| 12 | 04:04 | 03:59 | 03:46 | 04:04 | 04:13 | 03:58 |
| 13 | 03:50 | 03:35 | 03:42 | 03:35 | 03:38 | 03:48 |

The hardness and friability of the tablets produced from each of the thirteen runs were further characterized using a tablet tester according the USP Tablet Breaking Force Test and USP Friability Test. The observed hardnesses and friabilities are shown in Tables 17-18 below.

TABLE 17
Tablet Breaking Force (Hardness) (kp)

| | Compression Force (kN) | | | | | |
|---|---|---|---|---|---|---|
| Run # | 6 kN | 10.8 kN | 15.5 kN | 20.8 kN | 25 kN | 30 kN |
| 1 | 4.9 | 7.4 | 9.3 | 11.1 | 11.4 | 11.6 |
| 2 | 4.8 | 7.7 | 9.8 | 9.8 | 10.2 | 6.4 |
| 3 | 5.1 | 7.8 | 9.9 | 10.6 | 10.5 | 7.8 |
| 4 | 4.0 | 7.5 | 9.7 | 10.6 | 10.5 | n/a |
| 5 | 5.3 | 8.0 | 7.9 | 9.9 | 9.3 | 3.3 |
| 6 | 5.1 | 7.6 | 9.2 | 10.1 | 9.7 | 6.0 |
| 7 | 4.7 | 6.3 | 9.6 | 10.8 | 10.4 | 6.6 |
| 8 | 2.0 | 7.8 | 9.6 | 9.9 | 10.4 | 6.6 |
| 9 | 4.7 | 6.9 | 8.3 | 10.6 | 10.6 | 6.0 |
| 10 | 5.5 | 7.7 | 9.8 | 9.7 | 9.6 | 4.9 |
| 11 | 4.6 | 4.3 | 9.4 | 10.9 | 9.1 | 5.5 |
| 12 | 3.1 | 8.0 | 9.6 | 10.4 | 8.6 | 5.0 |
| 13 | 4.8 | 6.0 | 9.1 | 10.5 | 10.1 | 4.4 |

TABLE 18
Tablet Friability (%)

| | Compression Force (kN) | | | | | |
|---|---|---|---|---|---|---|
| Run # | 6 kN | 10.8 kN | 15.5 kN | 20.8 kN | 25 kN | 30 kN |
| 1 | 0.57 | 0.54 | 0.55 | 0.52 | 0.60 | 0.60 |
| 2 | 0.98 | 0.61 | 0.66 | 0.64 | 0.73 | 0.96 |
| 3 | 0.97 | 0.70 | 0.70 | 0.60 | 0.72 | 0.81 |
| 4 | n/a | 0.74 | 0.63 | 0.69 | 0.68 | n/a |
| 5 | 0.82 | 0.61 | 0.55 | 0.58 | 0.58 | 1.70 |
| 6 | 0.88 | 0.65 | 0.54 | 0.58 | 0.65 | 0.86 |
| 7 | 0.88 | 0.71 | 0.62 | 0.62 | 0.57 | 0.89 |
| 8 | 0.96 | 0.58 | 0.59 | 0.56 | 0.56 | 0.73 |
| 9 | 0.98 | 0.76 | 0.68 | 0.71 | 0.71 | 0.96 |
| 10 | 0.92 | 0.68 | 0.62 | 0.65 | 0.70 | 0.94 |
| 11 | 1.07 | 0.66 | 0.55 | 0.48 | 0.66 | 1.08 |
| 12 | 0.91 | 0.64 | 0.63 | 0.61 | 0.77 | 1.10 |
| 13 | 0.86 | 0.61 | 0.50 | 0.57 | 0.70 | 0.98 |

For comparison, the hardness, friability, and disintegration time of the commercially available naproxen sodium tablet prepared by wet granulation (as described in Example 1 above) were also determined. Tablets produced by the wet granulation process were found to have a hardness ranging from 6-16 kp, and a friability of 0.3%. The tablets were found to disintegrate in about 8 minutes in water.

Tablet dissolution profiles were determined for coated tablets from each of the thirteen runs tableted under 15.5 kN compression force. Each of the tablets from the thirteen runs were evaluated with two different coatings—Opadry® YS-1-4215 (“YS”) and Opadry® QX (“QX”). The tablet dissolution profiles were determined in accordance with the USP dissolution test (Apparatus 2, paddle, pH 7.8 phosphate buffer, at 37° C.±0.5° C.) (USP34-NF29 Chapter <711> Dissolution, Stage 6 Harmonization Bulletin dated Dec. 1, 2011; and Naproxen Sodium monograph USP41-NF36, Interim Revision Announcement dated May 1, 2018). Six tablets for each type of coating (six “YS” coated and six “QX” coated) were evaluated for each run and measured for percentage dissolution at each time point. The mean dissolution profiles (average of the six tablets for each time point) are shown in in Table 19 below for the two types of coated tablets and a commercially available tablet (Comparative Example Tablet, Table 2).

TABLE 19
Tablet Dissolution
(Percentage of naproxen sodium dissolved in phosphate buffer pH 7.4)

| | | Time (minutes) | | | | | |
|---|---|---|---|---|---|---|---|
| Run # | Coating | 0 min | 5 min | 10 min | 15 min | 20 min | 45 min |
| 1 | YS | 0 | 42 | 80 | 95 | 98 | 99 |
| | QX | 0 | 46 | 89 | 99 | 100 | 100 |
| 2 | YS | 0 | 43 | 82 | 97 | 100 | 100 |
| | QX | 0 | 40 | 82 | 100 | 102 | 102 |
| 3 | YS | 0 | 46 | 84 | 98 | 99 | 99 |
| | QX | 0 | 28 | 66 | 89 | 98 | 99 |
| 5 | YS | 0 | 45 | 81 | 95 | 99 | 99 |
| | QX | 0 | 38 | 77 | 96 | 100 | 101 |
| 6 | YS | 0 | 46 | 84 | 97 | 99 | 99 |
| | QX | 0 | 34 | 74 | 96 | 101 | 101 |
| 7 | YS | 0 | 49 | 83 | 97 | 100 | 100 |
| | QX | 0 | 36 | 75 | 96 | 100 | 101 |
| 10 | YS | 0 | 50 | 87 | 98 | 100 | 100 |
| | QX | 0 | 39 | 79 | 97 | 100 | 101 |
| 12 | YS | 0 | 45 | 83 | 98 | 101 | 102 |
| | QX | 0 | 33 | 74 | 95 | 101 | 103 |
| 13 | YS | 0 | 40 | 77 | 93 | 96 | 97 |
| | QX | 0 | 35 | 73 | 94 | 100 | 101 |
| Comparative | — | 0 | 32 | 64 | 86 | 95 | 101 |
| Example | | | | | | | |
| Tablet | | | | | | | |

### Example 4

### Preparation of Combination Bilayer Naproxen Sodium and Acetaminophen Tablet

The present example describes the preparation and disintegration profile of oral tablets comprising naproxen sodium granules in combination with acetaminophen in monolayer or bilayer forms. Various tablets comprising naproxen sodium or acetaminophen alone were also evaluated for their respective disintegration properties for comparison with the combination tablets.

As shown in this example, the combination of roller-compacted granules of naproxen sodium with acetaminophen in a monolayer tablet resulted in a significantly longer disintegration time as compared to the disintegration times observed for the tablets containing either of the individual active pharmaceutical ingredients—naproxen sodium or acetaminophen—alone. Unexpectedly, it was observed that a bilayer tablet comprising roller-compacted granules of naproxen sodium in one layer and acetaminophen in the second layer had a markedly shorter disintegration time than the monolayer tablet, and disintegrated more rapidly than the tablets comprising naproxen sodium alone.

### Part I—Evaluation of Combination Monolayer and Bilayer Tablets

Table 20 shows the composition of three tablet formulations prepared according to the present disclosure, including an oral tablet containing roller-compacted granules of naproxen sodium, a combination monolayer tablet containing roller-compacted granules of naproxen sodium with acetaminophen, and a combination bilayer tablet containing roller-compacted granules of naproxen sodium with acetaminophen in separate layers. The naproxen sodium granules employed in this Example were those prepared by roller compaction as generally described in Example 1 above; the granules for the present Example differed from those in Example 1 in that magnesium stearate was used as a lubricant in lieu of stearic acid. The naproxen sodium granules prepared had an average particle size for at least 90% of the particles (d90) of 1694 microns. Acetaminophen granules (comprising acetaminophen and starch) were obtained from a commercial vendor; the acetaminophen granules showed an average particle size d90 of 1537 microns.

The naproxen sodium tablet shown in Table 20 was prepared using the granules prepared in the present Example (with magnesium stearate) but according to the protocol described in Example 1.

For the combination monolayer and bilayer tablets, the same composition of roller-compacted granules as used for the naproxen sodium tablet was used for both combination tablets. The quantity of naproxen sodium granules incorporated for each tablet formulation was adjusted to the desired masses shown in Table 20; the naproxen sodium content provided by the granules was calculated based on the original composition and weight of granules added. The naproxen sodium content (150 mg) in the combination tablets was less than that of the single-active naproxen sodium tablet (220 mg), as the combination tablets were prepared for an intended single dose of two tablets.

The quantity of croscarmellose sodium was kept constant in the monolayer tablet and bilayer tablet to ensure that different quantity and type of superdisintegrant in either formulation would not influence the observed disintegration times. The quantity of other ingredients used in monolayer and bilayer tablets were kept constant to ensure that the impact of the excipients on disintegration was the same.

TABLE 20

| | Naproxen | | Combination | | Combination | |
|---|---|---|---|---|---|---|
| | Sodium | | Monolayer | | Bilayer | |
| | Tablet | | Tablet | | Tablet | |
| | Weight | % | Weight | % | Weight | % |
| Ingredients | (mg) | w/w | (mg) | w/w | (mg) | w/w |

Layer 1

| | | | | | | |
|---|---|---|---|---|---|---|
| Naproxen Sodium | 282.0^{&} | | 192.3* | 30.52 | — | — |
| Granules | | | | | | |
| Acetaminophen Granules | — | — | 364.4^{#} | 57.84 | 364.4 | 57.84 |
| Partial pregelatinized | — | — | 33.0 | 5.24 | 25.0 | 3.81 |
| starch | | | | | | |
| Croscarmellose sodium | — | — | 26.12 | 4.15 | 20.0 | 3.17 |
| Colloidal silicon dioxide | 10.40 | | 5.20 | 0.83 | 4.0 | 0.63 |
| Magnesium stearate | 18.0 | | 9.0 | 1.43 | 6.0 | 0.95 |
| Mannitol | 66.0 | | — | — | — | — |
| Sodium starch glycolate | 52.24 | | — | — | — | — |
| Total | | 100.0 | 630.0 | 100.0 | — | — |

Layer 2

| | | | | | | |
|---|---|---|---|---|---|---|
| Naproxen Sodium | — | — | — | — | 192.3* | 30.52 |
| Granules | | | | | | |
| Partial pregelatinized | — | — | — | — | 8.0 | 1.27 |
| starch | | | | | | |
| Croscarmellose sodium | — | — | — | — | 6.1 | 0.97 |
| Colloidal silicon dioxide | — | — | — | — | 1.2 | 0.19 |
| FD&C Blue No.1 | — | — | — | — | 1.0 | 0.16 |
| Magnesium stearate | — | — | — | — | 3.0 | 0.48 |
| Total tablet weight, mg | — | — | — | — | 630.0 | 100.0 |

^{&}equivalent to 220 mg naproxen sodium, 50 mg mannitol, 6 mg sodium starch glycolate, 3 mg colloidal silica, 3 mg magnesium stearate.
*equivalent to 150 mg naproxen sodium, 30 mg mannitol, 5 mg sodium starch glycolate, 2 mg colloidal silica, 2 mg magnesium stearate.
^{#}equivalent to 325 mg acetaminophen and 39.4 mg starch

TABLE 21

| | |
|---|---|
| Compression | Type of Combination Tablet |

| | | |
|---|---|---|
| Information | Monolayer | Bilayer |

| | |
|---|---|
| Equipment | FlexiTab |
| Tooling | Caplet shape, beveled edges, |
| | Type B, 17.5 × 7.4 mm punches |

| | | |
|---|---|---|
| Compression | 8 kN | Layer I- 1 kN, |
| Force (KN) | | Final - 8 kN |

Tablet Physical properties

| | | |
|---|---|---|
| Weight (mg) | 630 | 630 |
| Hardness (Kp) | 8.5 | 8.7 |
| Friability (%) | 0.1 | 0.1 |
| Thickness (mm) | 6.28 | 6.29 |
| Length (mm) | 17.14 | 17.16 |
| Width (mm) | 7.16 | 7.13 |
| Disintegration | 13 min 45 sec | 3 min 40 sec |
| time (min) | | |

The two combination tablet formulations described in Table 20 were compressed in a tablet press. The monolayer tablet was prepared using a single compression force of 8 kN; the bilayer tablet was prepared with an initial compression force of 1 kN for layer I (acetaminophen layer) and a final compression force of 8 kN after the addition of layer II (naproxen sodium layer) to form the bilayer tablet.

The hardness and friability of the tablets produced from each formulation were further characterized using a tablet tester according the USP Tablet Breaking Force Test and USP Friability Test. As shown in Table 21, the monolayer tablets exhibited similar hardness and friability. The tablets were also evaluated in accordance with USP disintegration test (USP43-NF38, Chapter <701> Disintegration, Stage 4 Harmonization Bulletin dated Apr. 26, 2019; uncoated tablet procedure, basket-rack assembly) in water in a basket-rack assembly with disks. As shown in Table 21, the combination bilayer tablet disintegrated within less than 4 minutes whereas the disintegration time observed for the monolayer tablet exceeded 10 minutes, nearly four times longer than the bilayer tablet disintegration time.

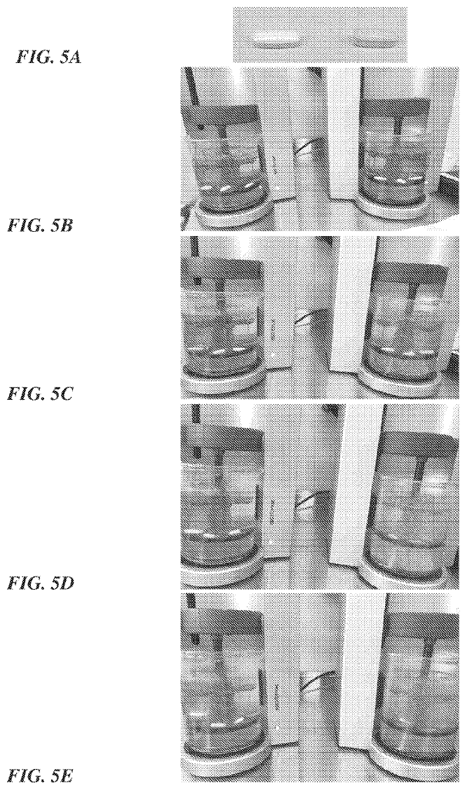

FIG. 5A shows a photograph of the monolayer tablet comprising the combination of naproxen sodium (roller-compacted granules) and acetaminophen (at left) and a bilayer tablet comprising naproxen sodium (roller-compacted granules) in one layer and acetaminophen in the second layer (at right). FIGS. 5B-5E show photographs illustrating the exemplary comparative disintegration of three tablets of each the monolayer combination tablet and the bilayer combination tablet over time. The photographs illustrate the time elapsed disintegration of each formulation in a disintegration apparatus at zero (0) seconds (FIG. 5B), at 10 seconds (FIG. 5C), at 35 seconds (FIG. 5D), and at 3 minutes, 3 seconds (FIG. 5E). The set-up and sample shown in the photographs were not used for the measurement of disintegration times provided in Table 21; the plastic disk required for the standard USP Disintegration Test was removed from the standard disintegration apparatus to improve visibility for photographic documentation. As shown in FIGS. 5B-5E and in the disintegration times collected in Table 21, the bilayer tablet comprising granulated naproxen sodium and acetaminophen in separate layers showed a significantly more rapid disintegration time as compared to the monolayer combination tablet.

Because the formula, shape, dimensions and other physical properties of monolayer and bilayer tablets were similar, the difference in disintegration times was attributed to the impact of naproxen sodium and acetaminophen matrix effect on the disintegration of the tablet. It was observed that the acetaminophen layer in the bilayer tablet burst out and disintegrated in 40 seconds while the naproxen sodium layer decreased in thickness and size and disintegrated in 3 minutes and 40 seconds. These observations suggested that acetaminophen disintegrated by burst release while naproxen sodium followed surface erosion mechanism of disintegration. On the other hand, monolayer tablet decreased in size and disintegrated in 13 minutes, 45 seconds.

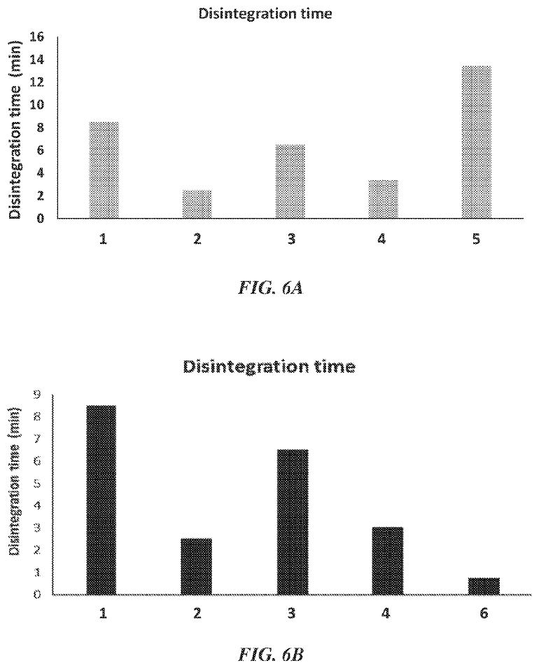

In a further comparison, the observed disintegration times were plotted against disintegration times measured for single-active formulations of naproxen sodium and acetaminophen, as determined by the USP Disintegration Test. FIG. 6A and 6B depict plots of the disintegration times for (1) a commercially available naproxen sodium tablet (comparative example as described in Table 2); (2) commercially available acetaminophen tablet (pregelatinized starch, powdered magnesium stearate, powdered cellulose, corn starch, sodium starch glycolate); (3) a tablet comprising roller-compacted granules of naproxen sodium (Naproxen Sodium Tablet described in Table 20); (4) the combination bilayer tablet comprising roller-compacted granules of naproxen sodium, and acetaminophen (Combination Bilayer Tablet in Table 20); (5) the combination monolayer tablet comprising roller-compacted granules of naproxen sodium, and acetaminophen (Combination Monolayer Tablet in Table 20); and (6) a half-layer tablet comprising acetaminophen (Layer I of the Combination Bilayer Tablet in Table 20, compressed at 1 kN).

As shown in plots of FIG. 6A and FIG. 6B, the disintegration time of the combination bilayer tablet was less than both disintegration times measured for the commercially available naproxen sodium tablet and the single-active tablet prepared using roller-compacted granules of naproxen sodium.

The reduction in disintegration time for the naproxen sodium in the bilayer tablet was attributed to the relative dimensions of the naproxen sodium tablets and the naproxen sodium half-layer in the bilayer tablet. The naproxen sodium half-layer in the bilayer tablet configuration has a thickness of 2.1 mm, whereas the commercially available standard naproxen tablet (entry #1) and the tablet comprising roller-compacted granules of naproxen sodium (entry #3) has a thickness of 4.3 mm. Following the rapid disintegration of acetaminophen in the bilayer tablet, the larger exposed surface area-to-volume ratio of the naproxen sodium half-layer relative to that of the tablet containing naproxen sodium alone was believed to contribute to the shorter disintegration time.

In contrast, the disintegration time for the monolayer combination tablet was observed to be much longer than the additive disintegration times of naproxen sodium alone (either traditional tablet or granulated) and acetaminophen alone. The increase in disintegration time for the monolayer tablet relative to the single-active tablets was attributed to matrix effects arising from the interaction of naproxen sodium and acetaminophen in the monolayer tablet.

What is claimed is:

1. A naproxen sodium tablet, comprising:
granules comprising naproxen sodium;
mannitol;
colloidal silicon dioxide;
one or more lubricants; and
one or more superdisintegrants,
wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.

2. A naproxen sodium tablet, comprising:
granules comprising naproxen sodium;
mannitol;
colloidal silicon dioxide;
stearic acid;
sodium starch glycolate; and
magnesium stearate,
wherein the tablet has a dissolution profile wherein at least 80% naproxen sodium is dissolved at 10 minutes and 100% naproxen sodium is dissolved at 20 minutes as determined by the USP apparatus-2 Dissolution Test in phosphate buffer pH 7.4 at 37° C.±0.5° C.

3. The naproxen sodium tablet of claim 1, wherein the tablet comprises 60-80% w/w naproxen sodium.

4. The naproxen sodium tablet of claim 1, wherein the naproxen sodium tablet comprises 10-20% w/w mannitol.

5. The naproxen sodium tablet of claim 1, wherein the granules comprise mannitol, colloidal silicon dioxide, stearic acid and sodium starch glycolate.

6. The naproxen sodium tablet of claim 1, wherein the granules are at least 85% w/w of the total weight of the naproxen sodium tablet.

7. The naproxen sodium tablet according to claim 1, wherein the naproxen sodium tablet comprises mannitol, sodium starch glycolate, and magnesium stearate as extragranular excipients.

8. The naproxen sodium tablet according to claim 1, wherein the naproxen sodium tablet comprises colloidal silicon dioxide as an extragranular excipient.

9. The naproxen sodium tablet according to claim 1, further comprising a film coating.

10. The naproxen sodium tablet of claim 1, wherein the naproxen sodium tablet has a disintegration time of less than 5 minutes as determined by the USP Disintegration Test in water using a basket-rack assembly with disks at 37° C.±0.5° C.

11. The naproxen sodium tablet according to claim 1, wherein the naproxen sodium tablet has a hardness between 2 and 14 kilopond (kp) as determined by tablet tester in accordance with the USP Tablet Breaking Force Test.

12. The naproxen sodium tablet according to claim 1, wherein the naproxen sodium tablet has a friability of less than or equal to 1% as determined by the USP Friability Test after 200 revolutions.

13. A method of treating pain or ache in a subject in need thereof, comprising administering a naproxen sodium tablet according to claim 1 to the subject.

14. The method of claim 13, wherein the pain or ache is associated with arthritis, muscular ache, backache, menstrual cramps, headache, toothache, or the common cold.

15. A method of reducing fever in a subject in need thereof, comprising administering a naproxen sodium tablet according to claim 1 to the subject.

16. A bilayer naproxen sodium tablet, comprising:
a naproxen sodium layer, comprising:
granules, comprising naproxen sodium;
mannitol;
colloidal silicon dioxide;
sodium starch glycolate;
starch and/or partially pregelatinized starch;
stearic acid or magnesium stearate; and
croscarmellose sodium, and
an acetaminophen layer, comprising:
acetaminophen;
colloidal silicon dioxide;
starch and/or partially pregelatinized starch;
stearic acid or magnesium stearate, and
croscarmellose sodium,
wherein the tablet has a disintegration time of less than 5 minutes as determined by the USP Disintegration Test in water using a basket-rack assembly with disks at 37° C.±0.5° C.

17. The bilayer naproxen sodium tablet of claim 16, wherein the tablet comprises between 100 and 200 mg naproxen sodium.

18. The bilayer naproxen sodium tablet of any claim 16, wherein the naproxen sodium tablet comprises 150 mg naproxen sodium.

19. The bilayer naproxen sodium tablet of claim 16, wherein the granules comprise mannitol, colloidal silicon dioxide, stearic acid or magnesium stearate, and sodium starch glycolate.

20. The bilayer naproxen sodium tablet of claim 16, wherein the granules comprising naproxen sodium are at least 25% w/w of the total weight of the tablet.

21. The naproxen sodium tablet according to claim 16, further comprising a film coating.

22. The naproxen sodium tablet of any claim 16, wherein the bilayer naproxen sodium tablet has a disintegration time of less than 4 minutes as determined by the USP Disintegration Test in water using a basket-rack assembly with disks at 37° C.±0.5° C.

23. The naproxen sodium tablet according to claim 16, wherein the naproxen sodium tablet has a hardness between 2 and 14 kilopond (kp) as determined by tablet tester in accordance with the USP Tablet Breaking Force Test.

24. The naproxen sodium tablet according to claim 16, wherein the naproxen sodium tablet has a friability of less than or equal to 1% as determined by the USP Friability Test after 200 revolutions.

25. A method of treating pain or ache in a subject in need thereof, comprising administering a bilayer naproxen sodium tablet according to claim 16 to the subject.

26. The method of claim 25, wherein the pain or ache is associated with arthritis, muscular ache, backache, menstrual cramps, headache, toothache, or the common cold.

27. A method of reducing fever in a subject in need thereof, comprising administering a bilayer naproxen sodium tablet according to claim 16 to the subject.
